(19) United States
(12) Patent Application Publication
(10) Pub. No.: US 2022/0249830 A1
(43) Pub. Date: Aug. 11, 2022

(54) Percutaneous Blood Pump Systems and Related Methods

(71) Applicant: HydraHeart, Inc., Woodinville, WA (US)

(72) Inventor: William R. Kanz, Woodinville, WA (US)

(73) Assignee: HydraHeart, Inc., Woodinville, WA (US)

(21) Appl. No.: 17/728,941

(22) Filed: Apr. 25, 2022

Related U.S. Application Data
(63) Continuation-in-part of application No. 16/748,787, filed on Jan. 21, 2020.
(60) Provisional application No. 63/178,640, filed on Apr. 23, 2021.
(60) Provisional application No. 62/794,988, filed on Jan. 21, 2019.

(51) Int. Cl. A61M 60/81; A61M 60/804; A61M 60/865; A61M 60/411

(52) U.S. Cl. CPC A61M 60/81; A61M 60/411; A61M 60/865; A61M 60/804

(57) ABSTRACT

A percutaneous trans-valvular blood pump system including a pump subsystem, catheter, and sheath is described. The pump subsystem includes a plurality of impeller pump assemblies arranged in tandem within a cannula portion of the catheter. The impeller pump assemblies are configured to operate in parallel so that blood pumped through one pump does not enter another pump.

### CROSS-REFERENCES TO RELATED APPLICATIONS

The present application is a non-provisional application claiming the benefit of priority under 35 U.S.C. § 119(e) from commonly owned and co-pending U.S. Provisional Application Ser. No. 63/178,640, filed on Apr. 23, 2021 and entitled “Percutaneous Blood Pump Systems and Related Methods,” and is a continuation-in-part of U.S. patent application Ser. No. 16/748,787, filed on Jan. 21, 2020 and entitled “Percutaneous Blood Pump Systems and Related Methods,” which claims the benefit of priority under 35 U.S.C. § 119(e) from commonly owned and co-pending U.S. Provisional Application Ser. No. 62/794,988 filed on Jan. 21, 2019 and entitled “Percutaneous Blood Pump Systems and Related Methods,” the entire contents of which are hereby incorporated by reference into this disclosure as if set forth fully herein.

### FIELD

The present invention relates generally to blood pumps and, more particularly, to an improved intra-vascular blood pump system and related methods.

### BACKGROUND

Blood pumps provide augmented blood flow rate for a damaged or diseased heart. Flow of blood pumps are limited by blood trauma (hemolysis) resulting from shear stress and transit time. Shear stress is affected by the diameter and rotational speed of the blood pump impeller. Percutaneous blood pumps are sized to be inserted through peripheral blood vessels. The diameter of a percutaneous blood pump is limited by the anatomy of the peripheral blood vessels. Prior art percutaneous blood pumps attempt to increase flow with expandable impellers which are technically difficult to implement reliably and safely.

Percutaneous trans-valvular blood pumps position their inlet cannula tip in a chamber of the heart. During high flow rates or when the blood volume adjacent to the inlet tip is low due to patient hemodynamic conditions for position of the inlet tip in the heart chamber, high negative pressure within the inlet cannula may result causing hemolysis through flow disturbances through the impeller or tissue damage due to high suction forces at the tip orifices.

Percutaneous blood pumps are inserted in peripheral vessels. The diameter of the blood pump is maximized to provide maximum flow while minimizing blood trauma. The blood pumps are used for many hours, even days. The introducer used to insert the blood pump in the vessel and establish hemostasis blocks the native flow through the vessel to the distal extremity. Prolonged blockage can lead to amputation. Blockage of flow reduces distal extremity pressure making vascular access difficult. The blood pumps are introduced into the body under emergency situations where time is critical, preventing adjunct procedures designed to ensure distal extremity perfusion prior to initiating circulatory support.

To access chambers of the heart, guide wire and catheters are used. For placement, the prior art utilizes the blood flow lumen from the tip though the non-rotating impeller and exits the impeller shroud blood port for placement of a guide wire and/or catheter. A guide wire is placed through this passage prior to insertion into the body then the blood pump is tracked over the guide wire for placement in the heart. However, if the inlet cannula of the blood pump becomes dislodged from the heart during treatment, the blood pump must be removed from the body to access the mentioned lumen for back-loading onto the guide wire used to safely access the heart. Prior art also utilizes a pig-tail catheter segment attached to the inlet cannula to aid in re-accessing the heart chamber in the event the inlet cannula becomes dislodged during use after the original guidewire is removed. The drawback is that the pig-tail catheter segment limits position location of the inlet cannula tip in the heart chamber and poses risks for cannula tip dislodgement or interference with valve function.

Other prior art utilizes a blood pump removably attached to the inlet cannula so access to the inlet tip may be accomplished without removal of the cannula from the body. This “over the wire” configuration does not require additional diameter beyond the diameter of the impeller to house the lumen for the guidewire/catheter access. However, removal of the blood pump is required increasing risk of contamination, bleeding, and infection.

The present invention is directed at overcoming, or at least improving upon, the disadvantages of the prior art.

### SUMMARY OF THE INVENTION

The present invention overcomes the limitations of prior art blood pumps by providing an improved intra-vascular blood pump having multiple impellers configured to increase flow rate. Impellers are arranged and rotationally driven in series with a trans-valvular cannula arranged for parallel trans-valvular flow through each impeller. Parallel flow results in summation of flow through each impeller for increased hemodynamic support for the patient with smaller insertion diameter for the physician. Optionally, the cannula is expandable to minimize pressure drop while being inserted in collapsed configuration similar to the size of an impeller. Rotational speed of all impellers is the same. Diameter of the impellers may be the same or progressively smaller allowing radial space for the expandable cannula in its collapsed configuration for insertion.

The present invention overcomes the limitations of prior art inlet cannula tips by providing an expandable structure configured to suspend the cannula inlet tip orifices away from the heart chamber tissue during use while being collapsed for insertion and removal.

The present invention overcomes the limitations of prior art introducers by providing a multi-lumen percutaneous blood pump introducer with access site bypass circuit configured to perfuse or drain the distal extremity after placement of the blood pump in the heart. The circuit allows blood flow through the annulus formed by the outer diameter of the blood pump drive sheath and the inner diameter of the introducer to a side-port of the introducer hemostasis valve through a connected catheter inserted percutaneously in the contralateral vessel which passes through introducer central lumen to side lumen having exit port in wall near introducer vessel access location to the distal vessel segment. Blood flow direction is dependent on anatomical placement. When placed in artery, blood flows into circuit through introducer tip under systemic pressure and exits circuit through catheter tip. When placed in vein, blood flows into circuit through catheter tip under systemic pressure and exits circuit through introducer tip.

The present invention overcomes the limitations of prior art access cannula systems by providing a lumen in the drive sheath of the blood pump configured to pass a removable guide sheath through a side-port proximal to the impeller crossing over the outer diameter of the impeller housing to access a lumen in the inlet cannula via a separate side-port distal to the impeller thereby bypassing the impeller region without adding additional diameter to the system beyond the size of the impeller housing. A guidewire may be passed through guide sheath to access the inlet cannula tip without removal of the blood pump from the body. The present invention provides for an “over the wire” type guide mechanism for selectively positioning and repositioning the intravascular blood pump and cannula at a predetermined location within the circulatory system of a patient without requiring removal of the blood pump from the patient.

In summary, the percutaneous blood pump system of the present invention boasts a variety of advantageous features, including but not limited to: An improved intra-vascular blood pump with multiple impellers and expandable cannula which provides the ability to produce increased flow rate at safe levels of blood trauma without increasing the diameter of the intravascular segments of the system compared to a single impeller blood pump; An expandable inlet cannula tip which provides the ability to prevent tip inflow occlusion when the tip is placed within anatomy that could block the tip inlet orifices; An introducer and distal extremity infusion catheter system to which provides the ability to bypass the insertion site obstruction and perfuse the distal extremity which the blood pump was introduced into the body; and a transvalvular percutaneous blood pump having one or more lumens which provide the ability to access the inlet cannula tip with guidewire or catheter for insertion and re-insertion into chambers of the heart without having to remove the blood pump from the body.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a side plan view of one example of a percutaneous blood pump system according to one embodiment of the disclosure;

FIG. 2 is a top plan view of an example of a distal end portion of the blood pump system of FIG. 1, comprising an inlet cannula with multiple impeller blood pumps according to one example embodiment;

FIG. 3 is a bottom plan view of the inlet cannula of FIG. 2;

FIG. 4 is a side plan view of the inlet cannula of FIG. 2;

FIG. 5 is a side sectional view of the inlet cannula of FIG. 2, taken along line 1-1 of FIG. 2;

FIG. 6 is a sectional view of the inlet cannula of FIG. 2, taken along line 2-2 of FIG. 4;

FIG. 7 is a sectional view of the inlet cannula of FIG. 2, taken along line 3-3 of FIG. 4;

FIG. 8 is a perspective view of an example of a first pump assembly forming part of the blood pump system of FIG. 1;

FIG. 9 is an exploded perspective view of the first pump assembly of FIG. 8;

FIG. 10 is a top plan view of the first pump assembly of FIG. 8;

FIG. 11 is a side plan view of the first pump assembly of FIG. 8;

FIG. 12 is a plan view of the distal end of the first pump assembly of FIG. 8;

FIG. 13 is a plan view of the proximal end of the first pump assembly of FIG. 8;

FIG. 14 is a bottom plan view of the first pump assembly of FIG. 8;

FIG. 15 is a sectional view of the first pump assembly of FIG. 8, taken along line 4-4 of FIG. 14;

FIG. 16 is a perspective view of an example of a second pump assembly forming part of the blood pump system of FIG. 1;

FIG. 17 is an exploded perspective view of the second pump assembly of FIG. 16;

FIG. 18 is a top plan view of the second pump assembly of FIG. 16;

FIG. 19 is a side plan view of the second pump assembly of FIG. 16;

FIG. 20 is a plan view of the distal end of the second pump assembly of FIG. 16;

FIG. 21 is a plan view of the proximal end of the second pump assembly of FIG. 16;

FIG. 22 is a bottom plan view of the second pump assembly of FIG. 16;

FIG. 23 is a sectional view of the second pump assembly of FIG. 16, taken along line 5-5 of FIG. 22;

FIG. 24 is a side plan view of an example of a proximal end portion of the blood pump system of FIG. 1, comprising a proximal impeller drive sheath, hub, and motor assembly according to one example embodiment;

FIG. 25 is a side plan view of an example of an introducer forming part of the blood pump system of FIG. 1;

FIG. 26 is a bottom plan view of the introducer of FIG. 25;

FIG. 27 is a detail view of a portion of the introducer of FIG. 25;

FIG. 28 is a cross-sectional view of the introducer of FIG. 25, taken along line 6-6 of FIG. 26;

FIG. 29 is a detail sectional view of a portion of the introducer of FIG. 25;

FIG. 30 is a side sectional view of an example of a bypass circuit using the introducer of FIG. 25 with the blood pump of FIG. 1;

FIG. 31 is a bottom plan view of an example of a distal tip of an inlet cannula of FIG. 2 having a tapered shape according to one embodiment;

FIG. 32 is a plan view of the distal tip of the inlet cannula of FIG. 31;

FIG. 33 is a side plan view of an example of a distal tip of an inlet cannula of FIG. 2 having a duckbill shape according to one embodiment;

FIG. 34 is a plan view of the distal tip of the inlet cannula of FIG. 33;

FIG. 35 is a side plan view of an example of a distal tip of an inlet cannula of FIG. 2 having an expandable balloon cage tip according to one embodiment;

FIG. 36 is a plan view of the distal tip of the inlet cannula of FIG. 35;

FIG. 37 is a side plan view of an example of a distal tip of an inlet cannula of FIG. 2 having an expandable mesh cage tip according to one embodiment;

FIG. 38 is a plan view of the distal tip of the inlet cannula of FIG. 37;

FIG. 39 is a side plan view of an example of a distal end portion of the blood pump system of FIG. 1, comprising an inlet cannula with multiple impeller blood pumps according to another example embodiment;

FIG. 40 is a section view of the inlet cannula of FIG. 39, taken along line 7-7 of FIG. 39;

FIG. 41 is a perspective view of the inlet cannula of FIG. 39 with a guide wire/catheter placed therethrough;

FIG. 42 is a side plan view of a portion of the inlet cannula of FIG. 39;

FIG. 43 is a side plan view of another example of a percutaneous blood pump system according to one embodiment of the disclosure;

FIG. 44 is a broken plan view of the blood pump system of FIG. 43;

FIG. 45 is an exploded plan view of the blood pump system of FIG. 43;

FIG. 46 is a plan view of an example of an introducer sheath forming part of the blood pump system of FIG. 43;

FIG. 47 is an exploded plan view of the introducer sheath of FIG. 46;

FIG. 48 is a sectional view of the introducer sheath of FIG. 46, taken along line A-A of FIG. 46;

FIG. 49 is an exploded plan view of an example of a catheter forming part of the blood pump system of FIG. 43;

FIG. 50 is a top plan view of the catheter of FIG. 49;

FIG. 51 is a sectional view of the catheter of FIG. 49, taken along line B-B of FIG. 50;

FIG. 52 is a top plan view of an example of an expandable cannula forming part of the catheter of FIG. 49;

FIG. 53 is a plan view of the distal end of the expandable cannula of FIG. 52;

FIG. 54 is a side plan view of the expandable cannula of FIG. 52;

FIG. 55 is a plan view of the proximal end of the expandable cannula of FIG. 52;

FIG. 56 is a bottom plan view of the expandable cannula of FIG. 52;

FIG. 57 is a side sectional view of the expandable cannula of FIG. 52, taken along line C-C of FIG. 52;

FIG. 58 is an exploded plan view of an example of a pump system forming part of the blood pump system of FIG. 43;

FIG. 59 is an exploded plan view of an example of a first pump assembly forming part of the pump system of FIG. 58;

FIG. 60 is an exploded plan view of an example of a second pump assembly forming part of the pump system of FIG. 58;

FIG. 61 is an exploded plan view of an example of a third pump assembly forming part of the pump system of FIG. 58;

FIG. 62 is a side plan view of the distal region of the pump system of FIG. 58, showing in particular the first, second, and third pump assemblies arranged in tandem;

FIG. 63 is a bottom plan view of the distal region of FIG. 62;

FIG. 64 is a section view of the distal region of FIG. 62, taken along ling H-H of FIG. 63;

FIG. 65 is a section view of the first pump assembly of FIG. 59, taken along line H-H of FIG. 63;

FIG. 66 is a section view of the third pump assembly of FIG. 61, taken along line H-H of FIG. 63;

FIG. 67 is a section view of the third pump assembly of FIG. 61, taken along line G-G of FIG. 62;

FIG. 68 is top plan view of a distal region of the blood pump assembly of FIG. 43;

FIG. 69 is a side sectional view of the distal region of FIG. 68, taken along line L-L of FIG. 68;

FIG. 70 is a side sectional view of a portion of the distal region of FIG. 68;

FIG. 71 is a side plan view of an obturator assembly forming part of the percutaneous blood pump system of FIG. 1;

FIG. 72 is a side plan view of the percutaneous blood pump system of FIG. 1 assembled in an insertion configuration according to one example embodiment;

FIG. 73 is a side section view of the assembled blood pump system of FIG. 72, taken along line P-P of FIG. 72;

FIG. 74 is a section view of the assembled blood pump system of FIG. 72, taken along lines N-N of FIG. 72;

FIG. 75 is a perspective view of another example of a pump subsystem according to one embodiment of the disclosure;

FIG. 76 is a perspective view of the distal end of the pump subsystem of FIG. 75, illustrating in particular first and second pump assemblies arranged in tandem;

FIG. 77 is a perspective view of a first pump assembly forming part of the pump subsystem of FIG. 75;

FIG. 78 is a partially exploded perspective view of the first pump assembly of FIG. 77, illustrating in particular the impeller assembly in exploded form;

FIG. 79 is another partially exploded perspective view of the first pump assembly of FIG. 77, illustrating in particular the bearing assembly in exploded form;

FIG. 80 is a partially exploded perspective view of the distal end of the pump subsystem of FIG. 76;

FIG. 81 is a top plan view of the distal end of the pump subsystem of FIG. 76;

FIG. 82 is a side sectional view of the distal end of the pump subsystem of FIG. 76, taken along line Q-Q in FIG. 81;

FIG. 83A is a detail view of highlight area R in FIG. 82, illustrating in particular a sectional view of the bearing assembly of FIG. 79;

FIG. 83B is a detail view of highlight area S in FIG. 82, illustrating in particular a sectional view of the impeller assembly of FIG. 78;

FIG. 83C is a sectional view of the bearing assembly of FIG. 79, taken along line 6-6 of FIG. 81;

FIG. 84 is a side plan view of the first pump assembly of FIG. 77;

FIG. 85 is an axial sectional view of the first pump assembly of FIG. 77, taken along line T-T in FIG. 84;

FIG. 86 is an axial sectional view of the first pump assembly of FIG. 77, taken along line U-U in FIG. 84;

FIG. 87 is an axial sectional view of the first pump assembly of FIG. 77, taken along line V-V in FIG. 84;

FIG. 88 is a perspective view of a second pump assembly forming part of the pump subsystem of FIG. 75;

FIG. 89 is an exploded perspective view of the second pump assembly of FIG. 88;

FIG. 90 is a top plan view of the second pump assembly of FIG. 88;

FIG. 91 is a side plan view of the second pump assembly of FIG. 88;

FIG. 92 is a side sectional view of the second pump assembly of FIG. 88, taken along line W-W in FIG. 90;

FIG. 93 is an axial view of the proximal end of the second pump assembly of FIG. 88;

FIG. 94 is an axial view of the distal end of the second pump assembly of FIG. 88;

FIG. 95 is an axial section view of the second pump assembly of FIG. 88, taken along line X-X of FIG. 90;

FIG. 96 is a side plan view of another example of an expandable cannula according to one embodiment;

FIG. 97 is a top plan view of the expandable cannula of FIG. 96;

FIG. 98 is a side sectional view of the expandable cannula of FIG. 96, taken along line Y-Y of FIG. 97;

FIG. 99 is a bottom plan view of the expandable cannula of FIG. 97;

FIG. 100 is a plan view of the distal end of the expandable cannula of FIG. 97;

FIG. 101 is a plan view of the proximal end of the expandable cannula of FIG. 97;

FIG. 102 is an axial sectional view of the expandable cannula of FIG. 97, taken along lines Z-Z of FIG. 99;

FIGS. 103-114 are plan views of various assembly configurations of the percutaneous blood pump system of FIG. 1, shown in order of method steps on using the system; and

FIG. 115 is a sectional view of a heart illustrating the percutaneous blood pump system of FIG. 1 in use;

FIG. 116 is side plan view of another example of a distal region of the blood pump assembly of FIG. 43 according to an alternative embodiment;

FIG. 117A is a side plan view of expansion area AA of FIG. 116;

FIG. 117B is a side sectional view of expansion area AA of FIG. 116;

FIG. 118 is an axial sectional view of the distal region of FIG. 116, taken along lines BB-BB of FIG. 117;

FIG. 119 is a side plan view of an expandable cannula forming part of the distal region of FIG. 116;

FIG. 120 is a bottom plan view of the expandable cannula of FIG. 119;

FIG. 121 is a top plan view of the expandable cannula of FIG. 119;

FIG. 122 is an axial sectional view of the expandable cannula of FIG. 119, taken along lines CC-CC of FIG. 119;

FIG. 123 is an axial sectional view of the expandable cannula of FIG. 119, taken along lines DD-DD of FIG. 119;

FIG. 124 is a side sectional view of the expandable cannula of FIG. 119, taken along lines EE-EE of FIG. 121;

FIG. 125 is a side sectional view of expansion area FF of FIG. 124;

FIG. 126 is side plan view of another example of a distal region of the blood pump assembly of FIG. 43 according to an alternative embodiment, illustrating in particular a catheter and pump assembly according to some embodiments;

FIG. 127 is a top plan view of the catheter and pump assembly of FIG. 126 according to some embodiments;

FIG. 128 is a side sectional view of the catheter and pump assembly of FIG. 126 taken along line FF-FF of FIG. 127 according to some embodiments;

FIG. 129 is a side plan view of an example of a cannula forming part of the catheter and pump assembly of FIG. 126 according to some embodiments;

FIG. 130 is an axial sectional view of the catheter and pump assembly of FIG. 126 taken along line GG-GG according to some embodiments;

FIG. 131 is an axial sectional view of the catheter and pump assembly of FIG. 126 taken along line HH-HH according to some embodiments;

FIG. 132 is a side plan view of an example of a pump assembly forming part of the catheter and pump assembly of FIG. 126 according to some embodiments;

FIG. 133 is a side plan view of an example of a shroud forming part of the pump assembly of FIG. 132 according to some embodiments;

FIG. 134 is an end view of the shroud of FIG. 133 according to some embodiments;

FIG. 135 is an end view of the pump assembly of FIG. 132 according to some embodiments; and

FIG. 136 is a side plan view of an example of a pump drive assembly forming part of the pump assembly of FIG. 132 according to some embodiments.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The percutaneous blood pump systems and related methods disclosed herein boasts a variety of inventive features and components that warrant patent protection, both individually and in combination.

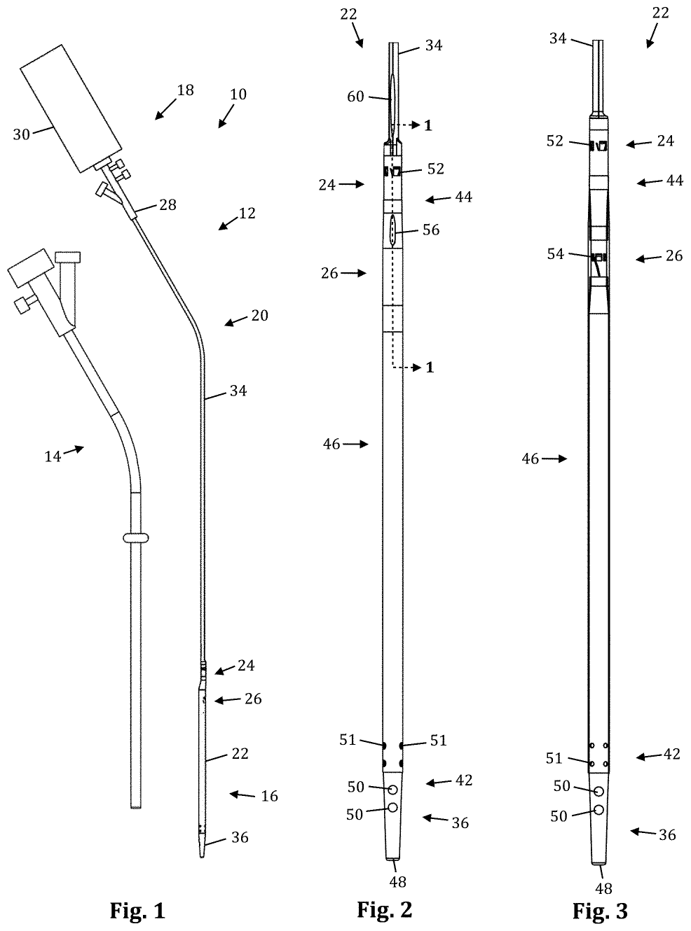

FIG. 1 illustrates an example of a percutaneous blood pump system 10 according to one embodiment of the disclosure. By way of example only, the blood pump system 10 of the present example includes a blood pump 12 configured to pump a volume of blood from one portion of a target site to another portion of a target site and an introducer 14 configured to facilitate the placement of the blood pump 12 within the target site. The blood pump 12 includes a distal end portion 16, a proximal end portion 18, and a middle portion 20 extending between the distal and proximal end portions. The distal end portion 16 comprises an expandable cannula 22 having a first or proximal pump assembly 24 and a second or distal pump assembly 26 arranged in series or tandem. The proximal end portion 18 comprises a drive hub 28 and drive motor assembly 30. The middle portion 20 comprises a flexible drive cable 32 contained within a drive cable sheath 34. The drive cable 32 connects the drive motor assembly 30 to the first and second pump assemblies 24, 26 and transfers rotational energy from the drive motor assembly 30 to the first and second pump assemblies 24, 26 to activate the pumps. The cannula 22 includes an inlet tip 36 that feeds into a plurality of separate and distinct internal lumens (by way of example only) providing inlet blood flow to each of the pump assemblies. In the instant example embodiment, a first lumen 38 provides a dedicated inlet flow into the first pump assembly 24 and a second lumen 40 provides a dedicated inlet flow into the second pump assembly 26 (See FIG. 5). In use, the blood pump 16 is a trans-valvular blood pump in which the inlet tip 36 of the multi-lumen cannula 22 is placed within a chamber of the heart (e.g. left ventricle) while outlet ports of the first pump assembly 24 and second pump assembly 26 are positioned in a trans-valvular manner (e.g. outlet ports of the proximal pump assembly 24 and distal pump assembly 26 are in the aorta on the opposite side of the aortic valve from the inlet tip 36) for providing left ventricular hemodynamic support.

By way of example only, two pump assemblies are shown. However, depending on the flow augmentation and insertion diameter required, additional pump assemblies may be configured. For example, as the desired insertion diameter of the system decreases, the number of pump assemblies may increase to achieve the same total flow augmentation amount and hemolysis index. Each pump assembly must have impeller blade design to produce a minimum amount of positive flow augmentation (e.g. >0.2 LPM average over the cardiac cycle) against physiological pressure differential between the cannula inlet and pump outlet (e.g. 60 mmHg average of the cardiac cycle).

FIGS. 2-7 illustrate an example of the inlet cannula 22 in greater detail. By way of example only, the inlet cannula 22 has a distal portion 42, a proximal portion 44, and a middle portion 46 extending between the distal and proximal portions. The distal portion 42 includes the inlet tip 36 comprising an axial aperture 48 at the distal tip of the cannula 22 and a plurality of large side apertures 50 and small side apertures 51 spaced about the distal portion 42. The axial aperture 48 and side apertures 50, 51 are configured to minimize pressure drop losses through the inlet tip and be tolerant of partial blockage by anatomical structures to maximize blood flow through first and second pump assemblies 24, 26. The proximal portion 44 connects to the distal end of the drive cable sheath 34 and includes a first or proximal set of egress apertures 52 configured for enabling flow of blood pumped through the first pump assembly 24 and a second or distal set of egress apertures 54 configured for enabling flow of blood pumped through the second pump assembly 26. Due to the structural configuration of linear or tandem pumps, the distal set of egress apertures 54 may be provided on only one side of the cannula 22, for example the bottom side. The proximal set of egress apertures 52 may be formed on any (or all) sides of the cannula 22. Egress apertures 52, 54 may include curved boundary surfaces that create a flow straightener or diffuser to recover rotational kinetic energy in the fluid exiting the impeller into pressure head energy thereby improving the efficiency of the pump and allowing for lower speeds and lower hemolysis for the same flow rate. The curved boundary surface may extend beyond the diameter of the pump housing and elongate into rib/blade features when released from the introducer. A guide wire aperture 56 positioned between the first and second pump assemblies 24, 26 (by way of example only) provides access to the guide wire lumen 58 extending distally within the cannula 22. A similar guide wire aperture 60 near the distal end of the drive cable sheath 34 allows the guide wire or a catheter (not shown) to bypass the proximal pump assembly 24 to access the inlet tip 36 of the cannula 22. Proximal access to the guide wire lumen 58 is via the hemostasis valve 136 on the drive hub 28, described below. The cannula middle portion 46 extends between the distal and proximal portions 42, 44 and includes a pair of distinct parallel lumens 38, 40 that fluidly connect the inlet tip 36 to the first and second pump assemblies 24, 26, respectively. The cannula middle portion 46 may be shaped straight or curved/angled for anatomical fit. In some embodiments, the cannula 22 may include only a single lumen that fluidly connects the inlet tip 36 to the first and second pump assemblies 24, 26.

As shown by way of example in FIG. 5, the proximal pump assembly 24 and distal pump assembly 26 are arranged in series or tandem and are driven by a drive cable 32 contained within a drive cable sheath 34. The blood flow into each pump assembly 24, 25 is accomplished within separate lumens 38, 40 of the multi-lumen cannula 22. Blood flows from the left ventricle of the heart (for example) into the cannula 22 through the inlet tip 36, passes through the second lumen 40 and into the distal pump assembly 26, and then exits the cannula 22 into the aorta (for example) through distal egress apertures 54 formed within the distal pump housing 104. Concurrently, blood also flows from the inlet tip 36 through the first lumen 38 and into the proximal pump assembly 24, and then exits the cannula 22 into the aorta (for example) through proximal egress apertures 52 formed within the proximal pump housing 66. Any single blood cell will only pass through one of the pump assemblies. Thus, the first and second pump assemblies 24, 26 are arranged in series (or tandem) but operate in parallel, enabling the blood pump 12 of the present example to pump twice the amount of blood compared to a single pump of the same size while exhibiting the same amount of hemolysis per volume pumped compared to a single pump of the same size.

The proximal pump assembly 24 and distal pump assembly 26 are connected to one another by way of a pump coupler 62. By way of example only, the pump coupler 62 is a flexible tube extending between the proximal and distal pump assemblies 24, 26 that contains the drive cable as it passes between the proximal and distal pumps, contains pressurized purge fluid for the distal pump(s) hydrodynamic bearings, and also allows the percutaneous blood pump 12 to be inserted through anatomy having a curved path, for example through a vein or artery. The cannula 22 may be constructed of flexible material (e.g. polyurethane, silicone) with resiliently elastic support material (e.g. Nitinol, nylon) or resiliently foldable frame material (e.g. laser cut stainless steel tubing) embedded in the wall or connected to the wall, which expands to operation configuration after being released from the confines of introducer sheath 14 then re-collapses to the confines of the introducer sheath 14 for removal from the patient.

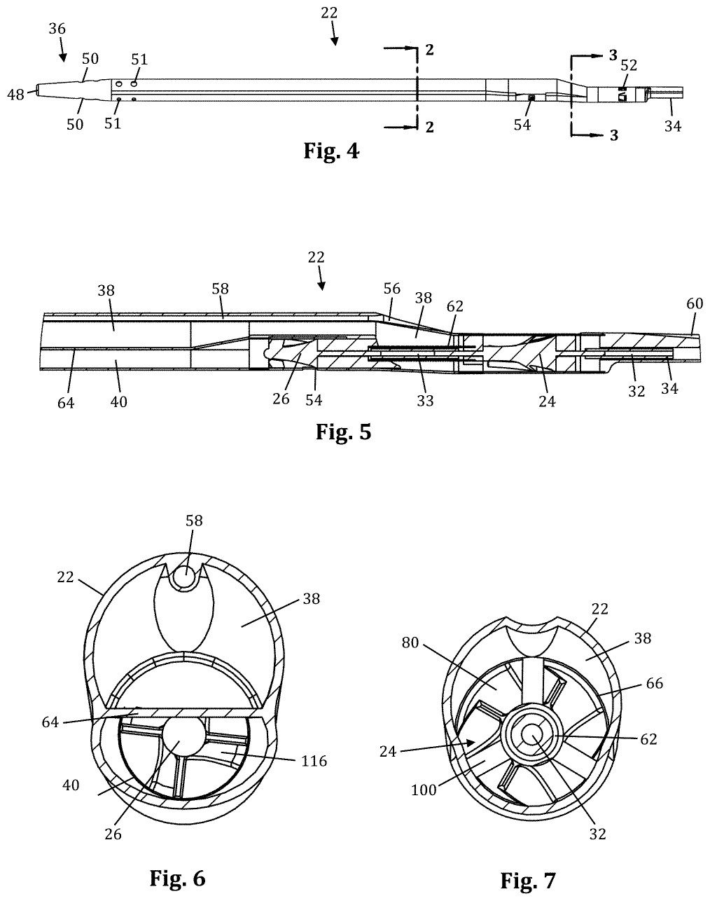

FIG. 6 illustrates a section view of the cannula 22 taken along lines 2-2 of FIG. 4, looking axially into the cannula 22 proximally toward the distal pump assembly 26. The first lumen 38 and second lumen 40 are separated by a septum 64 to enable the parallel flow arrangement. Each lumen cross-sectional area is sized to minimize and optimize pressure drop losses from the cannula inlet tip 36 to the pump housing 104 inlet.

FIG. 7 illustrates a section view of the cannula 22 taken along lines 3-3 of FIG. 4, looking axially into the cannula 22 proximally toward the proximal pump assembly 24. By way of example, the first lumen 38 at this point merges coaxial with the impeller 68 of the proximal pump assembly 24. As described below, the proximal pump assembly 24 includes a tip bearing 72 having radial support struts 100 to locate the tip bearing 72 inside the proximal pump housing 66 while allowing blood flow to pass between the radial support struts 100. The distal end of the impeller 68 couples with the drive cable 32 housed within the pump coupler 62 (see FIG. 5). The drive cable 32 transmits rotational energy from the drive motor assembly 30 to the proximal pump impeller 68 and the distal pump impeller 106. The pump coupler 62, made of flexible metal or plastic tubing, houses the rotating interpump drive cable 33 (see FIG. 5), connects the proximal pump tip bearing 72 to the distal pump shaft bearing 108 (see below), and transmits pressurized purge fluid for the hydrodynamic bearings of the distal pump(s).

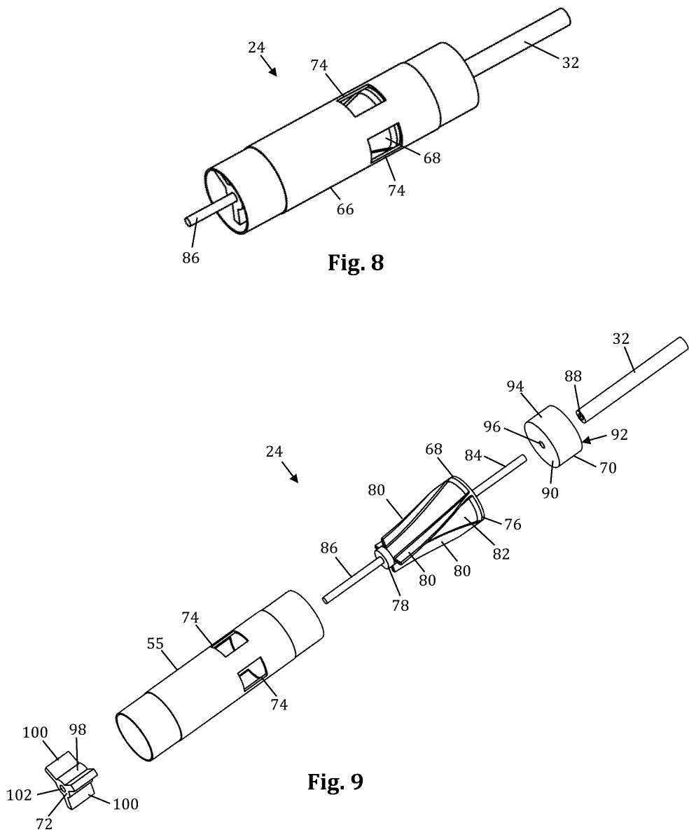

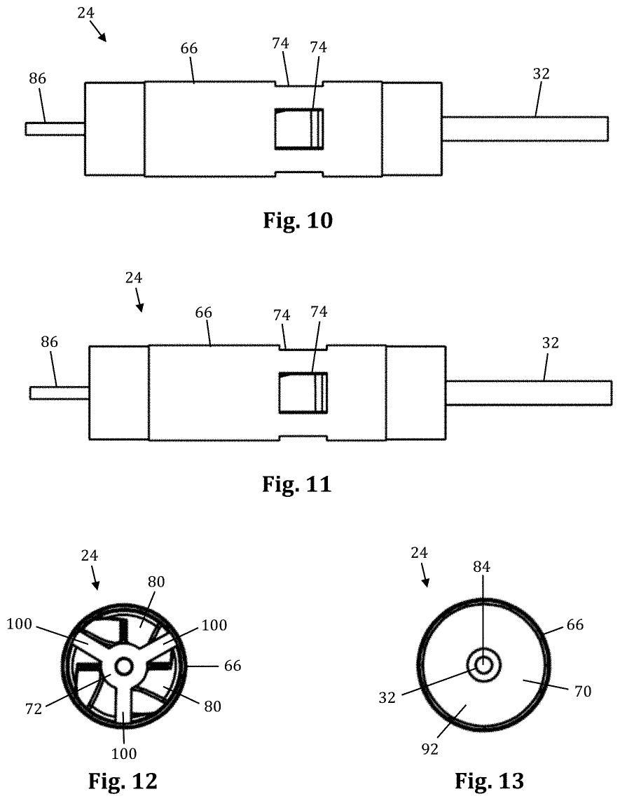

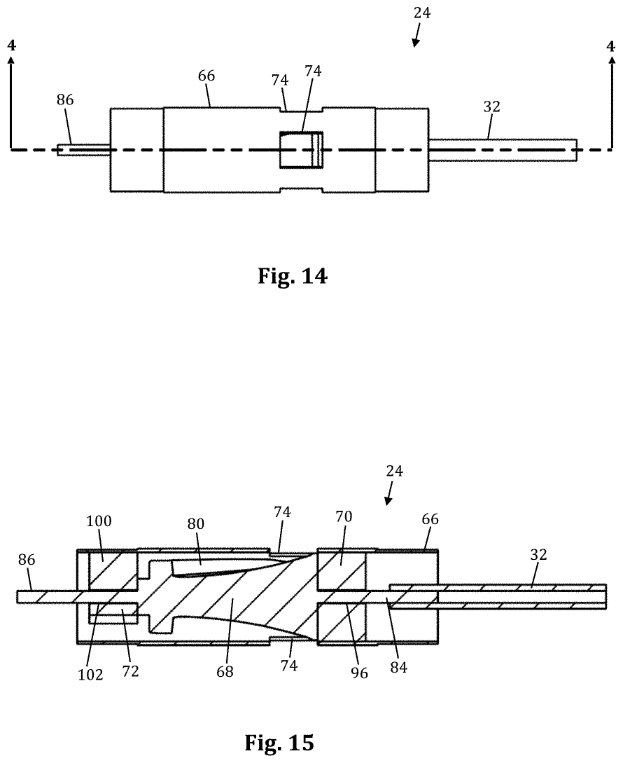

FIGS. 8-15 illustrate an example of the first or proximal pump assembly 24 in greater detail according to one embodiment. By way of example, the proximal pump assembly 24 includes a housing 66, an impeller 68, a shaft bearing 70, and a tip bearing 72. The housing comprises a generally cylindrical tube configured to contain the impeller 68, shaft bearing 70, and tip bearing 72 therein. The housing 66 has a plurality of egress apertures 74 formed therein in the proximity of the impeller 68 to enable blood flow out of the proximal pump assembly 24. The impeller 68 has a generally frustoconical shape including a base 76, a fulcrum 78, and a plurality of blades 80 (e.g. straight or curved) extending along the hub 82 from the base 76 to the fulcrum 78. The impeller 68 further includes a generally cylindrical shaft 84 extending proximally from the base 76 and a generally cylindrical post 86 extending distally from the fulcrum 78. The proximal shaft 84 is sized and configured to pass through the central aperture 96 of the shaft bearing 70 and engage the drive cable 32 directly as shown through inner lumen 88 or indirectly with a cylindrical coupler (not shown), thereby coupling the drive cable 32 to the impeller 68 so that the drive cable 32 may transfer rotational energy from the drive motor assembly 30 to the proximal pump impeller 68 to draw blood flow through the proximal pump assembly 24. The distal post 86 is sized and configured to pass through the central aperture 102 of the tip bearing 72 and engage the interpump drive cable 33 in the same manner, which couples with the proximal shaft 120 of the distal pump impeller 106, so that the drive cable 32 may transfer rotational energy from the motor drive motor assembly 30 to the distal pump impeller 106 by way of proximal pump impeller 68 and interpump drive cable 33 to draw blood flow through the distal pump assembly 26 at the same time (e.g. in parallel) as blood flow is being drawn through the proximal pump assembly 24. In addition, the proximal shaft 84, hub 82, and cylindrical post 86 may have an internal passage configured for transporting purge fluid to the distal pump(s). The frustoconical shape of the impeller 68 forces the blood to flow out of the egress apertures 74. This is known as a “mixed-flow” impeller design. Alternatively, the impeller 68 may have a hub 82 that is generally cylindrical in shape to create an “axial-flow” impeller, omitting the base 76 from the impeller 68. This would have a second tip bearing 72 on the proximal end of the impeller in place of shaft bearing 70. For the “axial-flow” design, the radial support struts 100 of the proximal tip bearing 72 may be configured with a curved shape to create a flow straightener or diffuser to recover rotational kinetic energy in the fluid exiting the impeller into pressure head energy thereby improving the efficiency of the pump allowing for lower speeds and lower hemolysis for the same flow rate. Alternatively, the pumps may have impellers of multi-stage design where blood passes through the multiple impellers in series, increasing pressure head performance allowing for further diameter reduction (e.g. <6 Fr).

The shaft bearing 70 is generally circular in shape and has a planar distal surface 90, a planar proximal surface 92, a curved radial outer surface 94, and a central aperture 96. The shaft bearing 70 is sized to fit snugly within the housing 66. The central aperture 96 is sized and configured to receive the proximal shaft 84 of the impeller 68 and allow the proximal shaft 84 and therefore the impeller 68 to rotate at high speed while maintaining axial alignment of the impeller 68 to ensure efficient rotation. The central aperture 96 may include one or more axial grooves to allow passage of pressurized purge fluid from the sheath 34 to the interface between the impeller base 76 and the bearing planar distal surface 90 to create a hydrodynamic bearing. The shaft bearing 70 may be comprised of two components a distal component and a proximal component with a compression spring element between them. The distal bearing outer surface 94 is sized for press-fit or adhesive bonding to the impeller housing preventing rotation while the proximal bearing is slip-fit on its outer surface 94 to allow axial translation with minimal radial run-out from the compression spring. The proximal surface 92 of the proximal shaft bearing is constrained from proximal axial movement by a shaft collar fixed to the rotating proximal shaft 84 and/or drive cable 32. The spring compression force is transmitted from the distal bearing through the spring to the “floating” but non-rotating proximal bearing to the rotating shaft collar and proximal shaft 84 to the impeller proximal surface 92 which is suspended on a thin-film of purge fluid (e.g. saline, dextrose solution) that is pressurized by the spring force reaction to the distal surface 90 of the distal bearing. This arrangement, or others providing the same functional effect as described below, reduces frictional heat between the rotating impeller and shaft bearing while minimizing the radial runout of the impeller at high speeds. Excessive heat from rotational friction is known to activate the clotting cascade which poses risk of vascular embolism to the patient. Excessive impeller runout can cause flow disturbances within the impeller flow region reducing pump efficiency, cause blood damage, or activate platelets. Instead of a separate compression spring element, the proximal shaft 84 may be hollow with lateral slits to form a rotating tension spring. This configuration would involve only one shaft bearing 70 and the shaft collar and the bearing load path would be through the shaft instead of the compression spring.

The tip bearing 72 has a base 98, a plurality of radial struts 100, and a central aperture 102 extending axially through the base. The radial struts 100 extend radially outward from the base 98 and are sized to span the distance between the base 98 and the housing 66 so that the tip bearing 72 may be sized and configured to fit snugly within the housing 66. The radial struts 100 may be straight or curved to form an inducer to precondition the fluid flow path to minimize hydraulic instability (e.g. flow separation, cavitation, vortices) within the impeller blade region. The central aperture 102 is sized and configured to receive the distal post 86 of the impeller 68 and allow the distal post 86 and therefore the impeller 68 to rotate at high speed while maintaining axial alignment of the impeller 68 to ensure coaxial rotation. Although shown in FIG. 9 by way of example only as having three radial struts 100, the tip bearing 72 may have any number of radial struts 100 without departing from the scope of the disclosure. In a similar manner to the hydrodynamic bearing arrangement described above for the proximal end of the impeller, the tip bearing may be fitted with a hydrodynamic bearing. In this instance, the shaft collar would be attached to the drive cable or impeller distal shaft and react the spring force on the distal face of the tip bearing while the fulcrum 78 would react the spring force on the proximal face of the tip bearing. In addition, the impeller housing would include anti-rotation and axial sliding feature to permit the tip bearing to self-align in the axial direction. Alternatively, proximal bearing and tip bearing may be comprised of blood immersed hydrodynamic bearings constructed from passive magnets for magnetic levitation of the pump shaft or of low friction materials (e.g. ruby, ceramic).

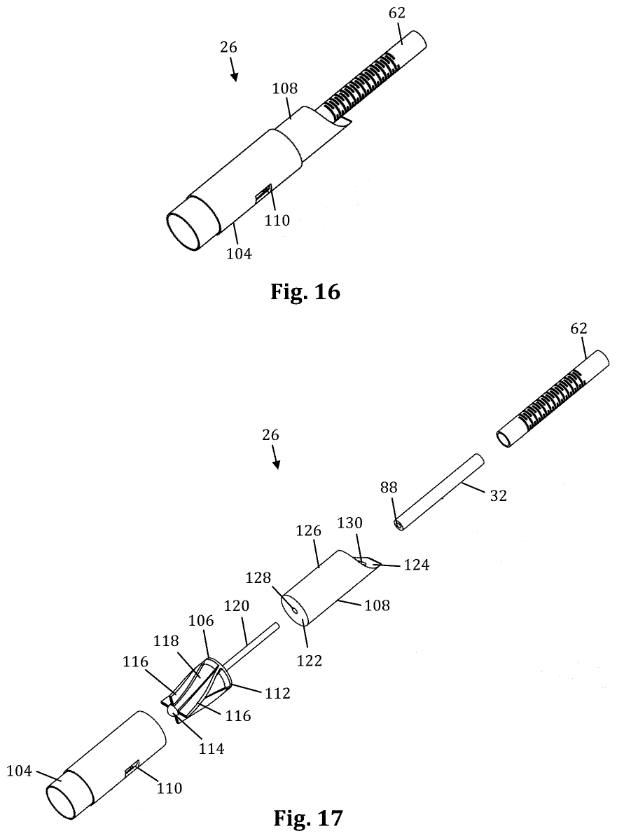

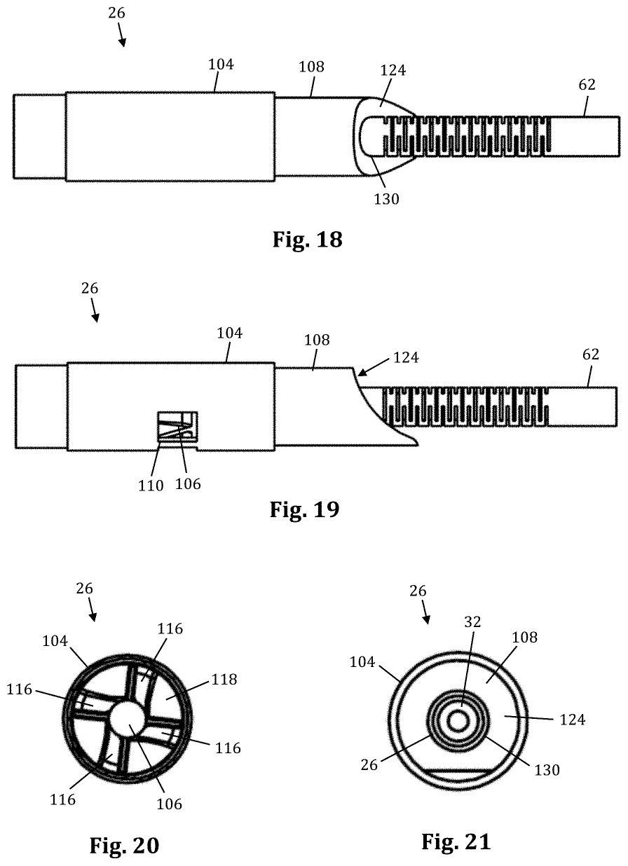

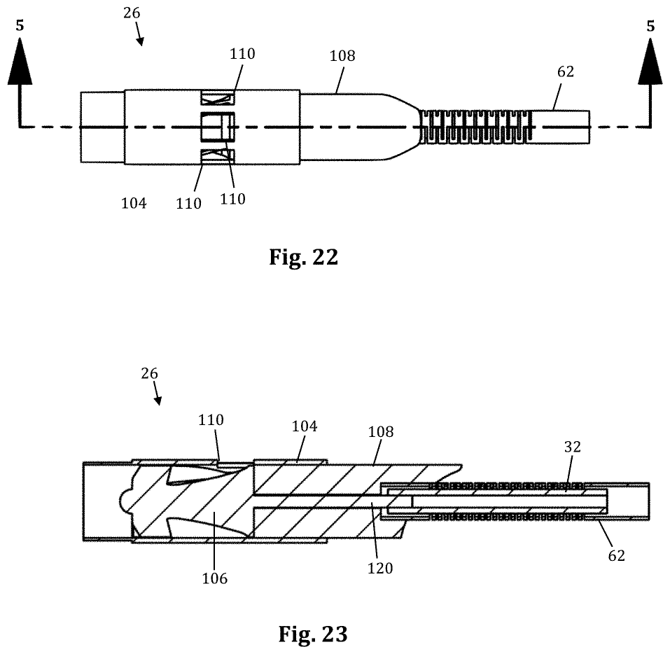

FIGS. 16-23 illustrate an example of the second or distal pump assembly 26 in greater detail according to one embodiment. By way of example, the distal pump assembly 26 includes a housing 104, an impeller 106, and a shaft bearing 108. The housing 104 comprises a generally cylindrical tube configured to contain the impeller 106 and shaft bearing 108 therein. The housing 104 has a plurality of egress apertures 110 formed therein in the proximity of the impeller 106 to enable blood flow out of the distal pump assembly 26. The impeller 106 has a generally frustoconical shape including a base 112, a fulcrum 114, and a plurality of blades 116 (e.g. straight or curved) extending along the sidewall 118 from the base 112 to the fulcrum 114. The impeller 106 further includes a generally cylindrical shaft 120 extending proximally from the base 112. The proximal shaft 120 is sized and configured to pass through the central aperture 128 of the shaft bearing 108 and engage an inner lumen 88 of the drive cable 32, thereby coupling the drive cable 32 to the impeller 106 so that the drive cable 32 may transfer rotational energy from the drive motor assembly 30 to the distal pump impeller 106 to draw blood flow through the distal pump assembly 26. The blades 116 are sized and configured to create a turbulence or current that draws the blood into the distal pump assembly 26. The frustoconical shape of the impeller 106 forces the blood to flow out of the egress apertures 110.

The shaft bearing 108 is generally cylindrical in shape and has a planar distal surface 122, a sloped proximal surface 124, a curved radial outer surface 126, and a central aperture 128. The shaft bearing 108 is sized to fit snugly within the housing 104. The central aperture 128 is sized and configured to receive the proximal shaft 120 of the impeller 106 and allow the proximal shaft 120 and therefore the impeller 106 to rotate at high speed while maintaining axial alignment of the impeller 106 to ensure efficient rotation. The sloped proximal surface 124 is configured to gently urge blood flow toward the proximal pump assembly 24. The proximal surface 124 may further include a generally cylindrical coupler recess 130 axially aligned with the central aperture 128 and configured to receive therein at least a portion of the pump coupler 62.

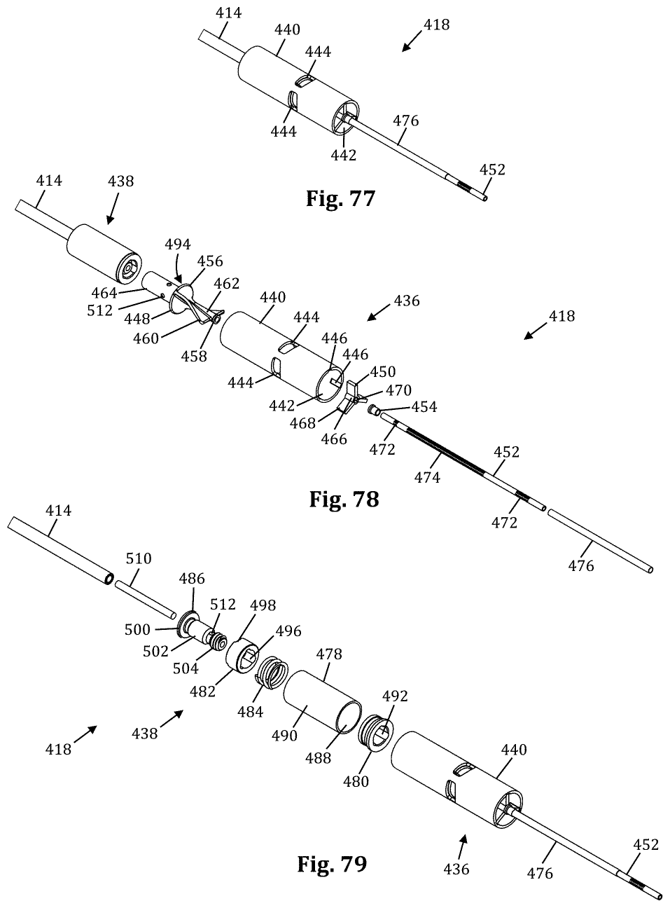

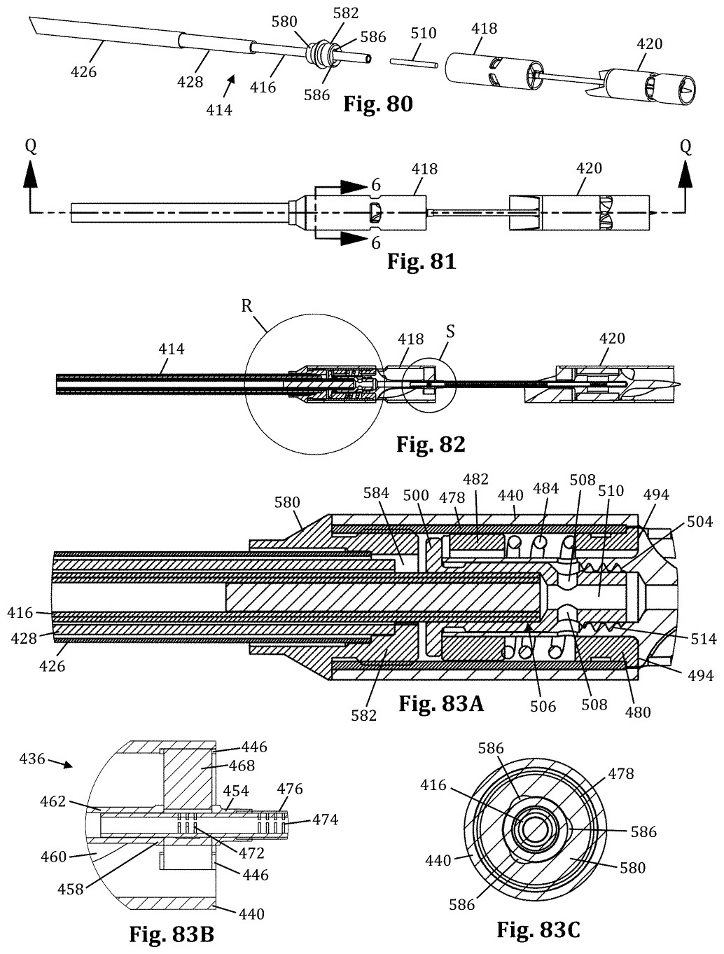

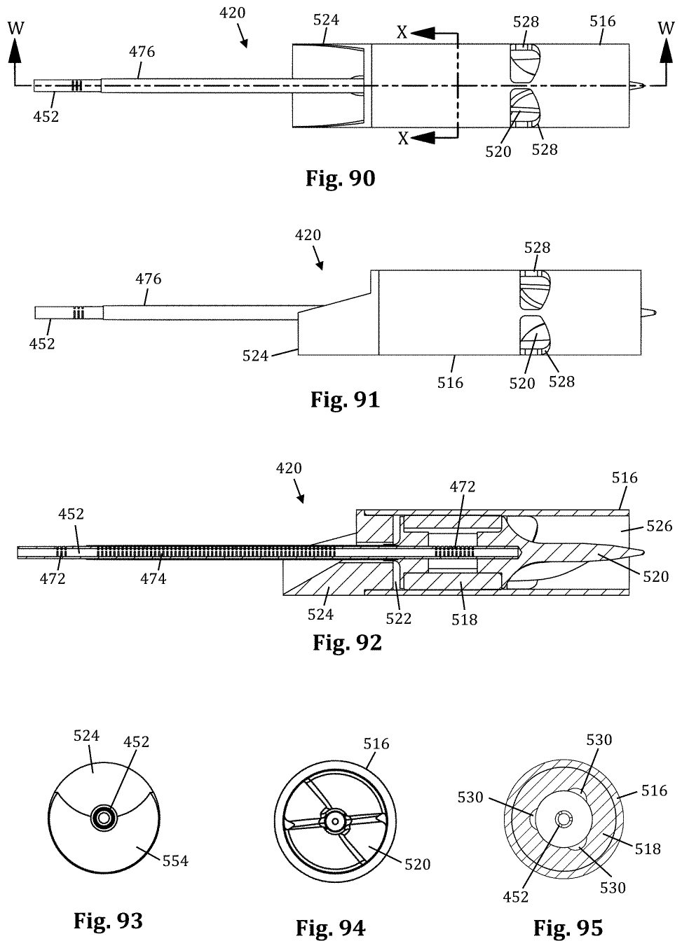

In some embodiments, the shaft bearing 108 (and/or any other bearing disclosed herein) may be a hydrodynamic bearing and blood seal. In such a case, the bearing may have axial slots inside the central aperture 128 to allow passage of purge fluid so the impeller 106 “hydroplanes” on bearing cooling interface to prevent thrombus formation and hemolysis. The impeller 106 may be spring loaded against the bearing 108 to create a rotating check valve for pressure and creating thin film for fluidic suspension of the impeller 106 on the bearing 108. See, e.g. FIGS. 77-95 below.

By way of example, the proximal and distal impellers 68, 106 are shown herein as fixed diameter components but can also be self-expanding flexible blades that are delivered in a folded or collapsed state inside a folded or collapsed pump housing inside the introducer sheath. Moreover, while the proximal and distal pump assemblies 24, 26 are shown herein as located at the proximal end 44 of the cannula 22, the proximal and distal pump assemblies 24, 26 may alternatively be located on the distal end 42 of the cannula 22, in which case the cannula 22 may not require resiliently strong support material in the wall because the pump outlet pressure may be sufficient to support the cannula wall from collapse.

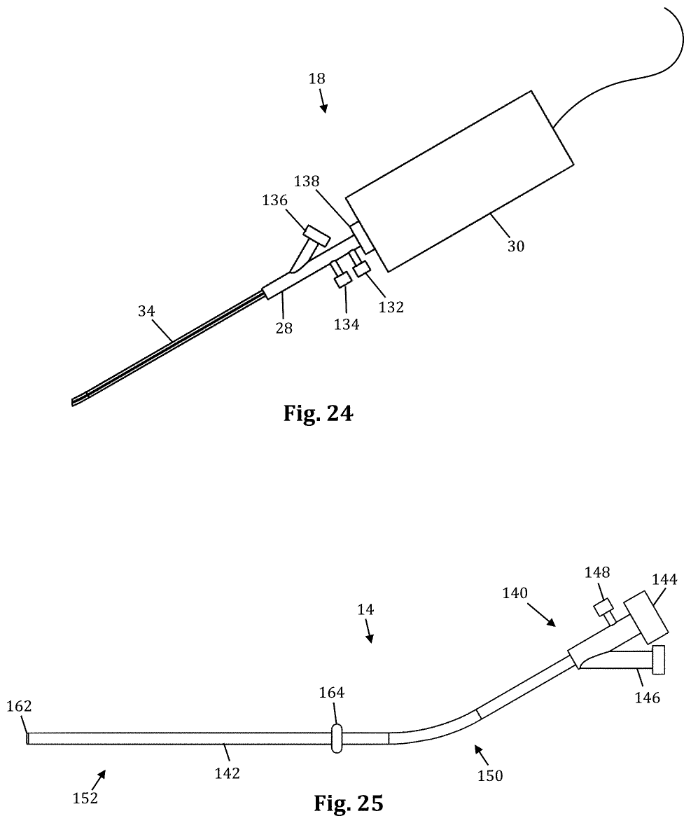

FIG. 24 illustrates an example of the proximal end portion 18 in greater detail according to one embodiment. By way of example, the proximal end portion 18 comprises a drive hub 28 and drive motor assembly 30. The drive hub includes a first port 132 providing access to a first lumen within the drive cable sheath 34, a second port 134 providing access to a second lumen within the drive cable sheath 34, and a hemostasis valve 136 for insertion of the guide wire. The drive motor assembly includes the drive motor (not shown) including a drive motor rotor located within the rotor housing 138.

By way of example, the drive cable sheath 34 comprises a flexible tubing of adequate length to locate the proximal pump assembly 24 in the desired anatomical position while the drive hub 28 is located outside the body. For example, in a typical transvalvular heart pump scenario, the blood pump 12 is advanced through the femoral artery accessed near a patient's groin such that one or more pumps (in this case proximal pump assembly 24 and distal pump assembly 26) is positioned in the aorta proximate the aortic valve. The drive cable sheath 34 houses the drive cable 32, which is made from multiple wires (filars) and layers for torque transmission and flexibility suitable for the anatomical route required. The drive cable 32 connects to a motor rotor (magnet) supported by bearings inside of the rotor housing 138. The first port 132 is fluidically connected to the central lumen of the drive cable sheath 34. The second port 134 is fluidly connected to a side lumen of the drive cable sheath 34. An infusion pump (not shown) may be fluidically connected to the second port 134. The infusion pump supplies the pump bearings (e.g. proximal pump shaft bearing 70, proximal pump tip bearing 72, and distal pump shaft bearing 108) with fluid (e.g. 30% dextrose intravenous solution) via the side lumen to de-air the system prior to insertion into a patient and to lubricate and flush the pump bearings during rotational operation. Return flow from the infusion pump travels along the central lumen and exits the through the first port 132 into a waste bag (not shown) while flushing wear particulate from the rotating drive cable 32 and drive cable sheath 34 interaction. The hemostasis valve 136 fluidically connects to another guide wire lumen 60 provided in the drive cable sheath 34 for passage of a guide wire sheath and guide wire to access the inlet tip 36 for selective positioning or repositioning of the cannula 22 in the heart.

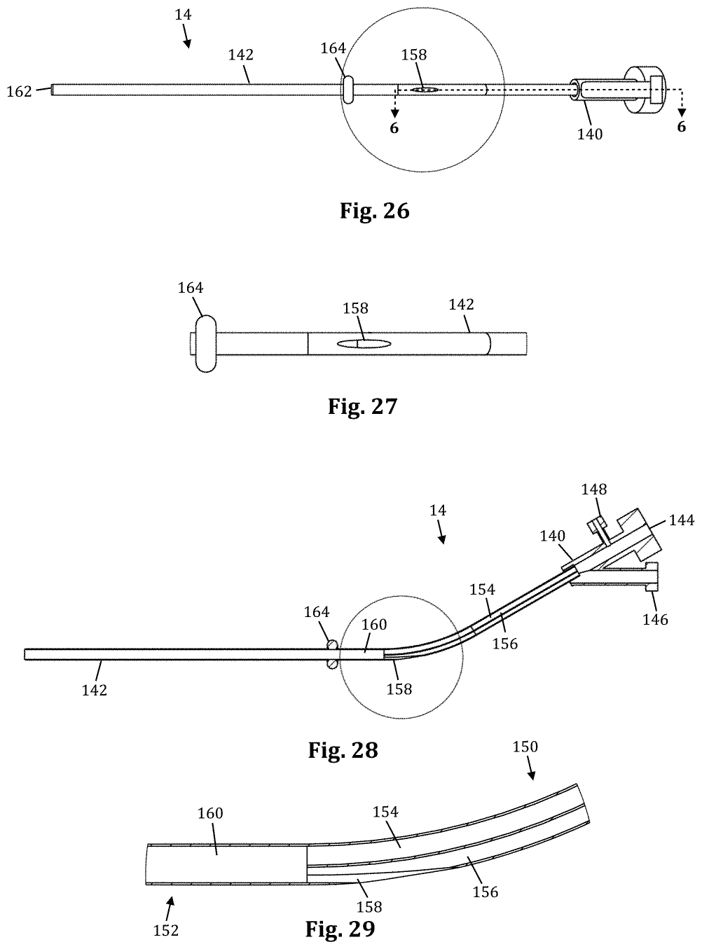

FIGS. 25-30 illustrate an example of an introducer 14 in greater detail according to one embodiment. By way of example only, the introducer 14 includes an introducer hub 140 locating at the proximal end of the introducer 14 and a flexible, thin-walled tubular sheath 142 extending distally from the hub 140. The introducer hub 140 comprises a first hemostasis valve 144, a second hemostasis valve 146, and a lumen port 148. The sheath 142 includes a proximal portion 150 and a distal portion 152. The proximal portion comprises a central lumen 154 and a side lumen 156, and a side lumen aperture 158 formed therein that provides access to the side lumen 156. The distal portion 152 includes a distal lumen 160, a tapered distal tip 162 designed for percutaneous insertion into a peripheral vessel, and an occlusion balloon 164 configured for sealing blood flow from the femoral artery around the introducer 14 to prevent bleeding at the introducer access site when the bypass catheter balloon 172 is inflated. By way of example only, the first hemostasis valve 144 may be of rotating collet or self-sealing duckbill-type valve for slidable sealing of the drive cable sheath 34 during selective positioning or repositioning of the cannula 22 in the heart. The second hemostasis valve 146 enables access to the proximal side lumen 156 of the sheath 142. The lumen port 148 provides access to the central lumen 154 of the sheath 142.

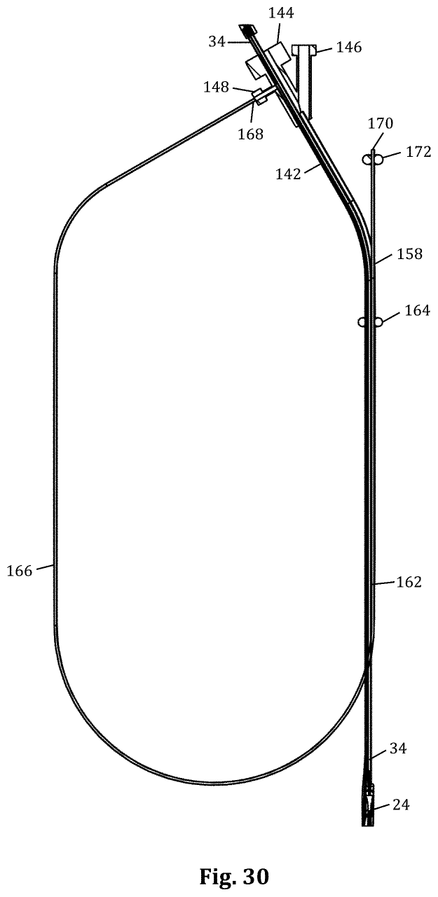

Referring now to FIG. 30, the side lumen aperture 158 forms a passage between the distal lumen 160 and the side lumen 156 for insertion of a distal perfusion cannula 166 to bypass blood under systemic pressure around the insertion site of the introducer sheath 142. The circuit allows blood flow through an annulus formed by the outer diameter of the drive cable sheath 34 and the inner diameter of the introducer sheath distal lumen 160, through the introducer sheath proximal central lumen 154 alongside the drive cable sheath 34, through the introducer sheath central lumen port 148, through removably connected distal perfusion cannula hub 168, through flexible tubing of the distal perfusion cannula 166 which has distal segment placed through the annulus formed by the outer diameter of the drive cable sheath 34 and the inner diameter of the introducer sheath distal lumen 160 then through the introducer sheath side lumen aperture 158 whereby the tip 170 of the distal perfusion cannula 166 is placed for flow distal to the insertion site of the introducer sheath 142 in the patient. Optionally, to isolate the insertion site from blood leakage from the patient, the introducer sheath 142 has occlusion balloon 162 connected to its outer diameter. The occlusion balloon 162 may be selectively inflated using saline injected through another side lumen of the introducer sheath 142 via side-arm (not shown) of introducer hub 140. Additionally, the distal perfusion cannula 166 may have a distal perfusion occlusion balloon 172 connected to its outer diameter, which may also be selectively inflated using saline injected through side-lumen (not shown) of distal perfusion cannula 166 via side-arm (not shown) of distal perfusion cannula hub 168.

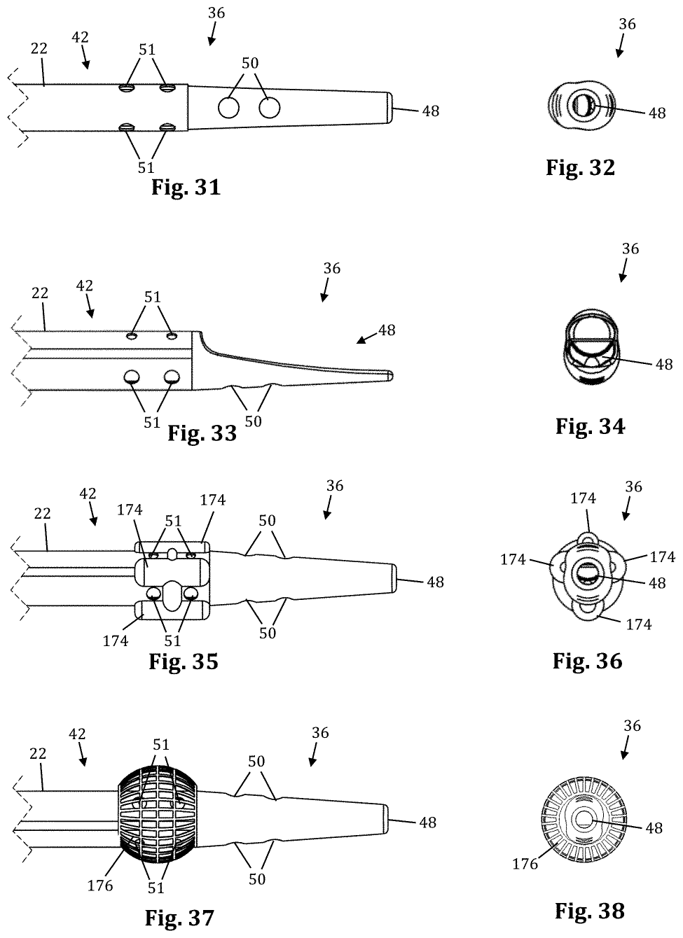

FIGS. 31-38 illustrate several examples of an inlet tip 36 that may be provided on the distal portion 42 of the cannula 22. By way of example, FIGS. 31-32 illustrate the inlet tip 36 example described above, comprising a tapered shape with an axial aperture 48 at the distal tip of the cannula 22 and a plurality of large side apertures 50 on the tapered portion and small side apertures 51 spaced about the distal portion 42 of the cannula proximal of the tapered portion. The axial aperture 48 and side apertures 50, 51 are configured to allow for sufficient blood flow into the cannula 22 to the first and second pump assemblies 24, 26.

FIGS. 33-34 illustrate an example of an inlet tip 36 comprising a duckbill shape similar to the taper shape of FIGS. 31-32 but with a section of the inlet tip 36 removed to create an extra large axial aperture 48 at the distal tip of the cannula 22. The inlet tip 36 also includes a plurality of large side apertures 50 on the tapered portion and small side apertures 51 spaced about the distal portion 42 of the cannula proximal of the tapered portion. The axial aperture 48 and side apertures 50, 51 are configured to allow for sufficient blood flow into the cannula 22 to the first and second pump assemblies 24, 26.

FIGS. 35-36 illustrate an example of an inlet tip 36 comprising a taper shape similar to the taper shape of FIGS. 31-32 with an expandable balloon element 174 provided thereon. The inlet tip 36 includes an axial aperture 48, a plurality of large side apertures 50 on the tapered portion, and a plurality of small side apertures 51 spaced about the distal portion 42 of the cannula proximal of the tapered portion. The axial aperture 48 and side apertures 50, 51 are configured to allow for sufficient blood flow into the cannula 22 to the first and second pump assemblies 24, 26. The expandable balloon element 174 is positioned about the side apertures 51 so that upon expansion of the balloon element 174 the side apertures 51 are protected from obstruction by tissue. The expandable balloon element 174 may be selectively inflated with saline after insertion into the patient by injecting saline through a side-port (not shown) in drive hub 28 which is connected to at least one side-lumen (not shown) in drive cable sheath 34.

FIGS. 37-38 illustrate an example of an inlet tip 36 comprising a taper shape similar to the taper shape of FIGS. 31-32 with an expandable mesh element 176 provided thereon. The inlet tip 36 includes an axial aperture 48, a plurality of large side apertures 50 on the tapered portion, and a plurality of small side apertures 51 spaced about the distal portion 42 of the cannula proximal of the tapered portion. The axial aperture 48 and side apertures 50, 51 are configured to allow for sufficient blood flow into the cannula 22 to the first and second pump assemblies 24, 26. The expandable mesh element 176 is positioned about the side apertures 51 so that upon expansion of the mesh element 176 the side apertures 51 are protected from obstruction by tissue. The expandable mesh element 176 may be made from elastic metal or plastic and is elastically collapsed inside of the introducer sheath 142 during insertion and then expands when the cannula 22 is slid outside of the introducer sheath 142 by pushing on the drive cable sheath 34. For removal from the patient, the mesh element 176 is slid back inside the introducer sheath 142 by pulling on the drive cable sheath 34.

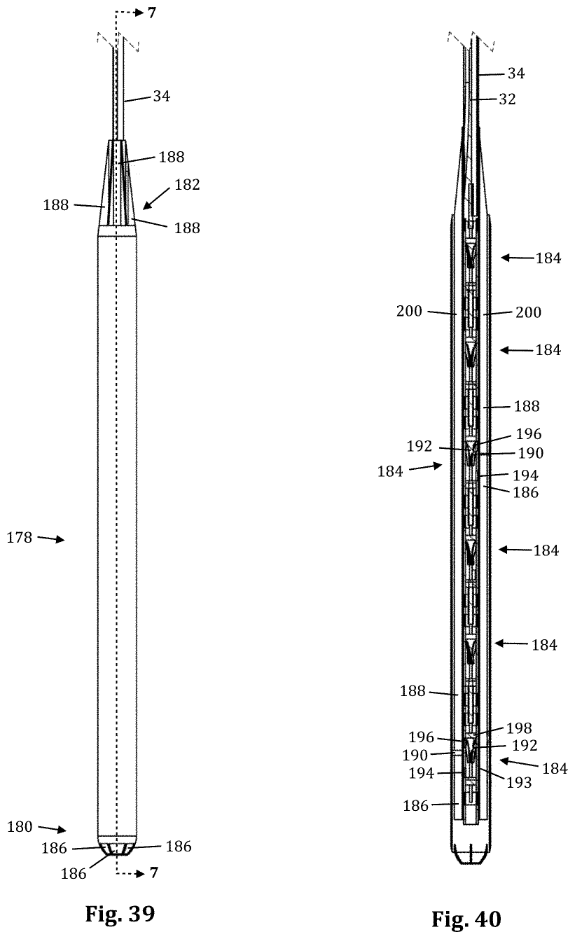

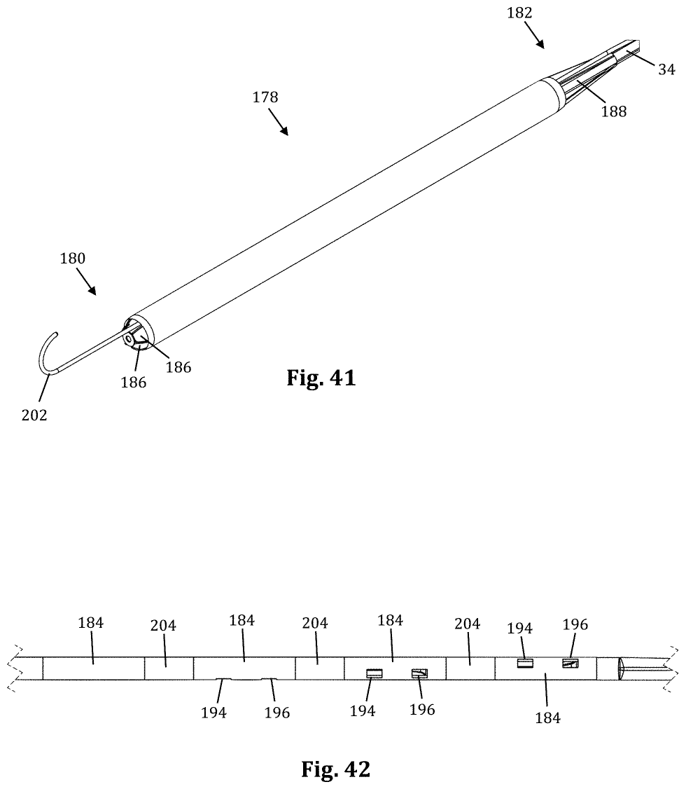

FIGS. 39-42 illustrate an example of a distal end portion 16 of the blood pump system 10, comprising an inlet cannula 178 with multiple impeller blood pumps according to one embodiment of the disclosure. By way of example only, the inlet cannula 178 of the present example comprises a radial multi-lumen cannula 178 having an inlet tip 180, an outlet 182, a plurality of pump assemblies 184 spaced along the inside of the cannula 178, and a plurality of inlet lumens 186 and outlet lumens 188 separated by lumen partitions 190. Each pump assembly 184 includes an impeller 192 contained within a dedicated pump housing 193, which includes an inlet port 194, an outlet port 196, and a shaft bearing 198 (similar to the pump arrangements described above). Each pump assembly 184 has a dedicated inlet lumen 186 supplying blood flow to the impeller 192 and a dedicated outlet lumen 188 for the blood to flow away from the impeller 192 to the outlet 182 where the blood enters the aorta (for example). Rotational energy from the drive cable (not shown) connected to an impeller 192 and supported by pump shaft bearing 198 creates a pressure difference between the inlet port 194 and the outlet port 196. The pressure at the outlet port 196 is higher than the pressure at the inlet port 194 resulting in flow from the inlet tip 180 to the outlet 182 due to the lumen partition 190. More specifically, blood flow travels from the inlet tip 180 into an inlet lumen 186 of the radial multi-lumen cannula 178, through an inlet port 194 of one of the several pump assemblies 184, through an outlet port 196 and into an outlet lumen 188 on the other side of a lumen partition 190 where it travels to the cannula outlet 182. The inlet port 194 and outlet port 196 may be arranged in the same angular plane or in different angular planes with respect to the central axis of the pump housing 193.

Each inlet lumen 186 and corresponding outlet lumen 188 (e.g. that are separated by a lumen partition 190) together form a radial channel 200. The radial channels 200 in the radial multi-lumen cannula 178 may be arranged in a linear orientation with respect to the central axis of the pump housing 193 or alternatively in a spiral orientation. In some embodiments, the lumen partition 190 may be of self-sealing type having construction that allows through passage of a tubular or wire structure such as a catheter or guide wire 202 and seals against retrograde flow from the proximal side when the tubular or wire structure is removed. In some embodiments, the lumen partition 190 may be an elastically expandable orifice or other type of hemostasis valve. Alternatively, the lumen partition 190 may be constructed to allow a guide wire to remain in place while allowing the guide wire or catheter tip to selectively be positioned. The radial multi-lumen cannula 178 may be of expandable/collapsible construction in which it is inserted in the patient constrained within a smaller diameter introducer sheath 142 (for example) and then self-expands by way of elastic support members in the wall of the tubing when selectively positioned outside the introducer sheath 142 by pushing on the drive cable sheath 34. Removal from the patient may be by way of selectively withdrawing the drive cable sheath 34 to position radial multi-lumen cannula 178 inside a smaller diameter introducer sheath 142 causing elastic support members used in construction of radial multi-lumen cannula 178 to collapse. As shown in FIG. 42, the plurality of pump assemblies 184 are arranged in series and separated by pump assembly couplers 204, but operate in parallel as with the pump assemblies described above.

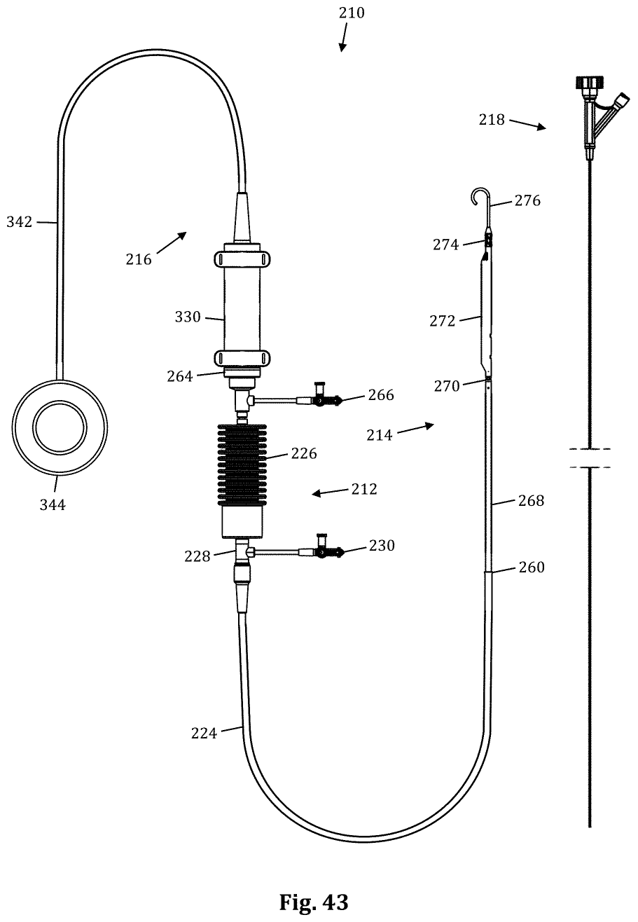

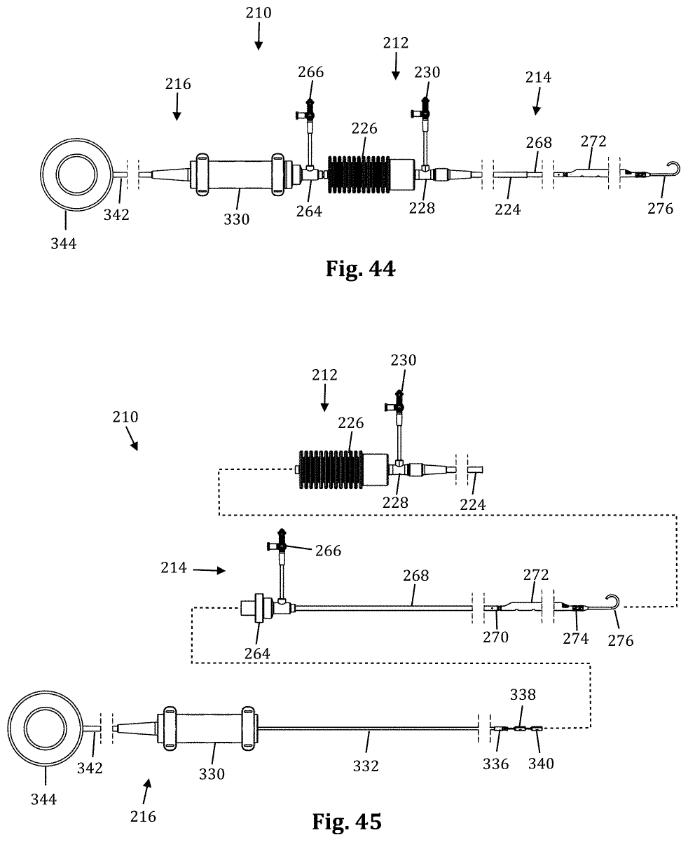

FIGS. 43-45 illustrate an example of a percutaneous blood pump system 210 according to another embodiment of the disclosure. By way of example only, the blood pump system 210 of the present example includes a sheath 212, a catheter 214, a pump subsystem 216, and an obturator 218. Generally, the sheath 212 is configured to receive the catheter 214 therein and constrain the expandable cannula 272 in a collapsed configuration during insertion while sealing the catheter shaft 268 for positioning in an unsheathed state in the body. The catheter 214 functions as a conduit for blood flow from the heart chamber into the body, and also seals the guide wire 202 and pump drive shaft 334 for insertion into the body. The pump subsystem 216 creates a pressure difference between the inlet and outlet apertures of the cannula to drive blood flow into the body.

The blood pump system 210 of the present example is similar to the blood pump system 10 of the previously described example in that the blood pump system 210 is a multiple impeller pump system having a plurality of pump assemblies arranged in a linear or tandem arrangement but operating in parallel, in that blood (or any other fluid) pumped through one pump assembly will not pass through any other pump assemblies. However, the blood pump system 210 of the present example differs from the blood pump system 10 described above in at least two aspects: first, the blood pump system 210 of the present example employs a single lumen cannula that supplies all of the pump assemblies with intake blood, and second, the pump subsystem 216 of the present example is removable/replaceable and is inserted after initial placement of the catheter and removal of the guide wire 202 and obturator 218. This enables the use of a smaller diameter catheter than may be otherwise needed.

The blood pump system 210 of the present example is scalable to meet the needs of any particular patient. For example, the number of pump assemblies may be increased or decreased depending on flow requirements without affecting hemolysis efficiency. If a smaller catheter is needed (for example due to partial blockage, other anatomical limitations, or to reduce access site bleeding complications), then additional pump assemblies may be added to increase flow with the same hemolysis index (mg plasma free hemoglobin per liter blood pumped). If lower hemolysis index is needed, then additional pump assemblies may be added and the pump speed of each reduced, resulting in the same flow with lower hemolysis index.

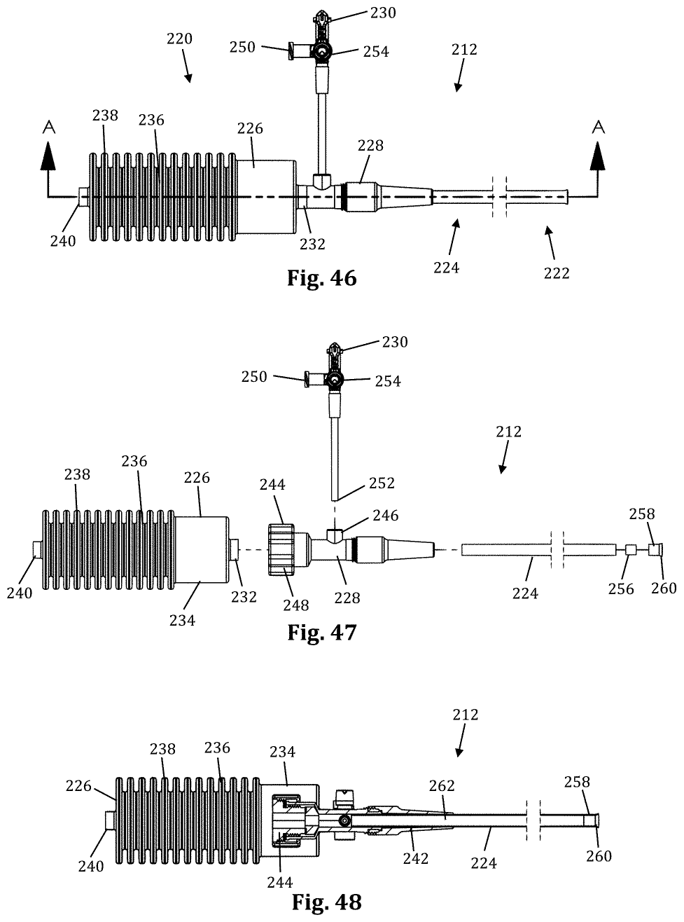

FIGS. 46-48 illustrate an example of a sheath 212 in greater detail according to one example embodiment. Continued reference to FIGS. 43-45 may be made to understand how the various components of the sheath 212, catheter 214, and pump subsystem 216 interact with one another. By way of example only, the sheath 212 includes a proximal end 220, a distal end 222, and a shaft 224 extending between the proximal and distal ends 220, 222. The proximal end 220 may include a sterile sleeve 226, a hemostasis valve 228, and a fluid line 230. The sterile sleeve 226 of the instant example comprises a clear, thin-walled plastic sleeve having a distal seal 232 configured to fluidly seal the hemostasis valve 228 proximal of the fluid line 230, a distal chamber 234 configured to contain the valve handle 244 within a sterile environment while allowing the valve handle 244 rotational freedom within the distal chamber 234, an expandable chamber 236 having bellowed folds 238 configured to enable the expandable chamber 236 to expand proximally to cover a length of the catheter shaft 268 (see e.g. FIGS. 72-73), and a proximal seal 240 configured to fluidly seal the catheter shaft 268 during use. The sterile sleeve 226 is configured to maintain the sterility of the hemostasis valve 228 and the catheter shaft 268 while repositioning the catheter 214 relative to the sheath 212 after insertion into the patient during an initial sterile procedure.

The hemostasis valve 228 of the present example embodiment is a clear, rigid polymer valve assembly with an elastomeric seal and a rotating locking handle that seals blood inside the patient while also allowing axial translation of the catheter shaft 268 within the sheath 212. The hemostasis valve 228 includes (by way of example only) an inner lumen 242 extending axially therethrough, a proximally-located rotating valve handle 244 and a fluid port 246 fluidly connected to and extending laterally from the inner lumen 242. The lumen 242 is sized and configured to allow passage of a number of instrument and components therethrough, including but not limited to the catheter shaft 268, obturator 218, pump drive shaft 334, and the like, and is also configured to allow the flow of fluids therethrough. The valve handle 244 may be generally cylindrical in shape and have a friction element 248 (e.g. grooves, ridges, etc.) to enable a user to grip and rotate the valve handle 244 through the sleeve 226 to selective close and open the hemostasis valve 228. The fluid port 246 fluidly connects to the outlet opening 252 of the fluid line 230. By way of example only, the fluid line 230 is a clear flexible polymer tube having a proximal inlet opening 250, distal outlet opening 252, and a stopcock valve 254. The fluid line 230 may be configured to allow de-airing and flushing of the hemostasis valve 228 and sheath 212 with anticoagulant fluid (for example).

By way of example, the distal end 222 comprises a tip tube 256 and a tip funnel 258. The tip tube 256 is a thin-walled rigid tube positioned within the tip funnel 258 and transmits forces applied to the catheter 214 for sheathing and unsheathing of the expandable cannula 272. The tip funnel 258 guides the expandable cannula 272 into the shaft 224 and may include an outwardly-flared edge 260 that flexes and collapses when inserted into a patient's vasculature. The shaft 224 is a generally cylindrical flexible tube having an inner lumen 262 extending therethrough. The shaft 224 may be sized and configured such that the outer diameter of the proximal end fits snugly within the inner lumen 242 of the hemostasis valve 282 so as to fluidly seal the interface between the outer shaft 224 and hemostasis valve 228. The inner lumen 262 is sized and configured to allow passage of a number of instruments and components therethrough, including but not limited to the catheter shaft 268, obturator 218, pump drive cable assembly 332, and the like, and is also configured to allow the flow of fluids therethrough.

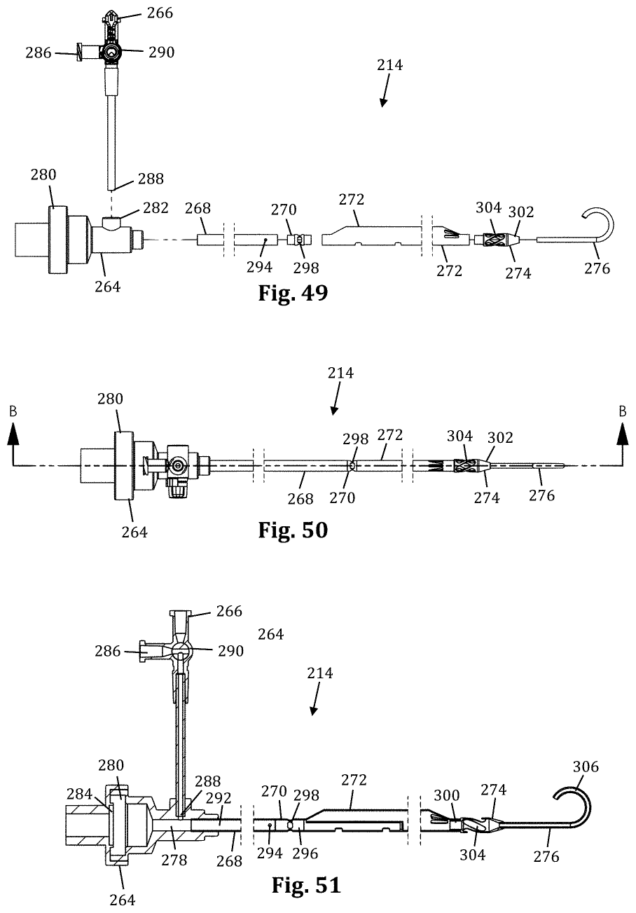

FIGS. 49-51 illustrate an example of a catheter 214 in greater detail according to one example embodiment. Continued reference to FIGS. 43-45 may be made to understand how the various components of the sheath 212, catheter 214, and pump subsystem 216 interact with one another. By way of example only, the catheter 214 comprises a hemostasis valve 264, a fluid line 266, a shaft 268, a proximal shroud 270, an expandable cannula 272, a catheter tip housing 274, and an atraumatic tip 276. The hemostasis valve 264 of the present example embodiment is a clear, rigid polymer valve assembly with an elastomeric seal and a pump latch that seals blood inside the patient while also providing a conduit for and allowing axial translation of the guide wire 202 and pump shaft 334 within the catheter 214. The hemostasis valve 264 includes (by way of example only) an inner lumen 278 extending axially therethrough, a pump latch 280, and a fluid port 282 fluidly connected to and extending laterally from the inner lumen 278. The lumen 278 is sized and configured to allow passage of a number of instrument and components therethrough, including but not limited to the obturator 218, guide wire 202, pump shaft 334, and the like, and is also configured to allow the flow of fluids therethrough. The pump latch 280 is configured to interact with a corresponding latching element of the pump motor assembly 330 and includes a locking element 284 (e.g. snap-fit, etc.) to securely connect the catheter hemostasis valve 264 to the pump 216 while simultaneously opening the hemostasis valve 264. In some embodiments, the pump latch 280 may be configured to provide visual, audible, and/or tactile feedback to the user to indicate a successful association has been made. The fluid port 282 fluidly connects to the outlet opening 288 of the fluid line 266. By way of example only, the fluid line 266 is a clear flexible polymer tube having a proximal inlet opening 286, distal outlet opening 288, and a stopcock valve 290. The fluid line 266 may be configured to allow de-airing and flushing of the hemostasis valve 264 and catheter 214 with anticoagulant fluid (for example). The fluid line 266 also provides an access portal for connecting a pressure monitoring system (not shown) to measure the patient blood pressure on the outside of the catheter 214 near the proximal shroud 270.

The shaft 268 by way of example only comprises an elongated thin-walled, flexible tubular member extending between the hemostasis valve 264 and the proximal shroud 270. The shaft 268 has an outer diameter configured for snug interaction within the lumen 278 of the hemostasis valve 264 so as to provide a sealed interface between the hemostasis valve 264 and the catheter shaft 268. The shaft 268 further includes an inner lumen 292 sized and configured to allow passage of a number of instruments and components therethrough, including but not limited to the obturator 218, guide wire 202, pump shaft 334, and the like, and is also configured to allow the flow of fluids therethrough. The shaft 268 also includes at least one distal opening 294 positioned near the interface with the proximal shroud 270, the distal opening 294 configured to enable a pressure monitoring system to measure the patient blood pressure on the outside of the catheter 214 near the proximal shroud 270.

The proximal shroud 270 by way of example only is a generally cylindrical tubular member of rigid construction having an inner lumen 296 extending axially therethrough and one or more flow ports 298 formed therein. The proximal shroud 270 is positioned between the distal end of the catheter shaft 268 and the proximal end of the expandable cannula 272, and serves as a housing for the proximal pump impeller 356 and thus the flow ports 298 serve as inlet ports or outlet ports for impeller flow depending on flow direction. The inner lumen 296 is sized and configured to allow passage of a number of instruments and components therethrough, including but not limited to the obturator 218, guide wire 202, one or more pump assemblies 336, 338, 340, and the like, and is also configured to allow the flow of fluids therethrough.

The catheter tip housing 274 by way of example only is a generally cylindrical rigid member having a inner lumen 300 extending axially therethrough, a tapered distal tip 302, and a plurality of flow ports 304 formed therein. The inner lumen 300 is sized and configured to allow passage of a number of instruments and components therethrough, including but not limited to the obturator 218, guide wire 202, and the like, and is also configured to allow the flow of fluids therethrough. The tapered distal tip 302 provides a tapered transition into the patient's vasculature. The flow ports 304 serve as inlet ports or outlet ports for blood flow to or from the cannula 272 depending on flow direction. The flow ports 304 may be curved to prevent blockage by anatomical structures inside the heart.

The atraumatic tip 276 by way of example only is a flexible member having an inner lumen 306 extending from the distal end of the catheter 214. The inner lumen 306 is sized and configured to allow passage of a number of instruments and components therethrough, including but not limited to the obturator 218, and guide wire 202. When configured for left-ventricular support (for example), the atraumatic tip 276 prevents trauma to heart by flexing and distributing axial load along larger area. The atraumatic tip 276 also positions the catheter tip housing 274 away from structures in heart that may impede blood flow into the cannula 214, and provides conduit for tracking the catheter over the guide wire 202 for positioning in the heart.

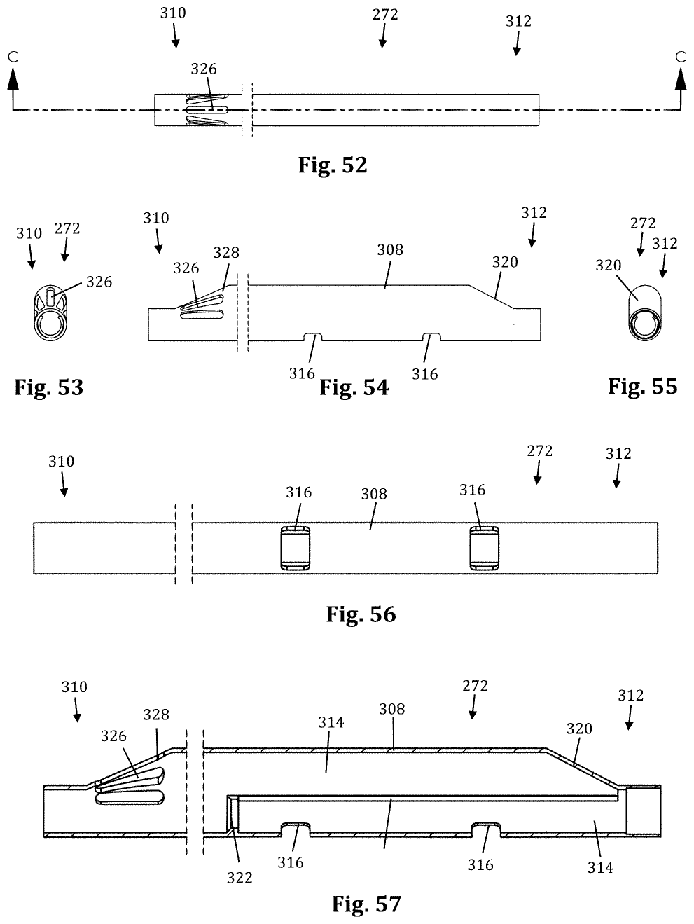

FIGS. 52-57 illustrate an example of an expandable cannula 272 in greater detail according to one embodiment. Continued reference to FIGS. 43-45 may be made to understand how the various components of the sheath 212, catheter 214, and pump subsystem 216 interact with one another. The cannula 272 of the present example comprises a thin-wall, self-expanding, re-collapsible, cylindrical tube providing a conduit for blood flow out of heart (for example). The cannula 272 is inserted in collapsed configuration (see, e.g. FIG. 74) and expands to an expanded operating configuration upon emergence from the sheath 212. The expanded cross-section is configured to be suitable for the desired length of the cannula 214, number of tandem pumps operating, and desired maximum pressure drop from the cannula inlet to the pump inlets at the desired maximum system flow rate.

By way of example only, the expandable cannula 272 comprises an expandable body 308, a distal end 310, a proximal end 312, inner lumen 314, and one or more flow port(s) 316 formed in the bottom side of the body 308 (by way of example). The expandable body 308 comprises a thin-walled, self-expanding, re-collapsible tube made from flexible polymer and reinforcing frame, and includes an inner lumen 314 extending axially therethrough and a proximal taper 320 on the outer proximal surface of the expandable body 308. The inner lumen 314 is sized and configured to allow passage of a number of instrument and components therethrough, including but not limited to the obturator 218, guide wire 202, and the like, and is also configured to allow the flow of fluids therethrough.

The inner lumen 314 also houses one or more middle and/or distal pump assemblies 338, 340 that are inserted into the cannula 272 after expansion of the expandable body 308. A flow port 316 is provided for each middle and/or distal pump assembly 338, 340 within the cannula 272 to allow the fluid to flow through tandem arranged impellers (e.g. impellers 374, 396) in parallel through the cannula 272. To ensure that the pump assemblies are properly aligned with the flow ports 316 upon insertion into the cannula 272, the inner lumen may further include one or more pump alignment features, including but not limited to (and by way of example only) a laterally-oriented pump stop 322 and/or an axially oriented pump guide 324. By way of example only, the pump stop 322 may be a physical barrier to prevent advancement of the pump assemblies once the shroud flow ports 384 are laterally aligned with the cannula flow ports 316. The pump guide 324 of the present example comprises an elongated axially-oriented tongue or rail in the inner lumen 314 that is configured to slidably mate with a complementary alignment feature 386 (e.g. a corresponding axially-aligned groove or track) formed on the outer surface of the middle and/or distal pump shroud(s) 372 to ensure rotational alignment of the flow ports 316, 384.

The proximal taper 320 facilitates collapsing of the expandable body 308 for removal from the body. More specifically, to remove the expandable cannula 272 from the body, a user exerts an axial force in the proximal direction to pull the catheter back through the sheath 212. As the proximal taper 320 encounters the tip funnel 258 of the sheath 212, the proximal taper 320 translates the axial force applied to the cannula body 308 by the tip tube 256 (due to its rigidity) into inward radial force to collapse the expandable body 308 for removal through the sheath 212.

The distal end 310 is configured with a plurality of apertures 326 formed in a distal taper element 328 of the expandable body 308. The apertures 326 may function as ingress or egress apertures (depending of flow direction) to the cannula 272, augmenting the cross-sectional area of the catheter tip housing 274. The cannula 272 also provides a conduit for tracking the catheter 214 over the guide wire 202 for positioning in the heart.

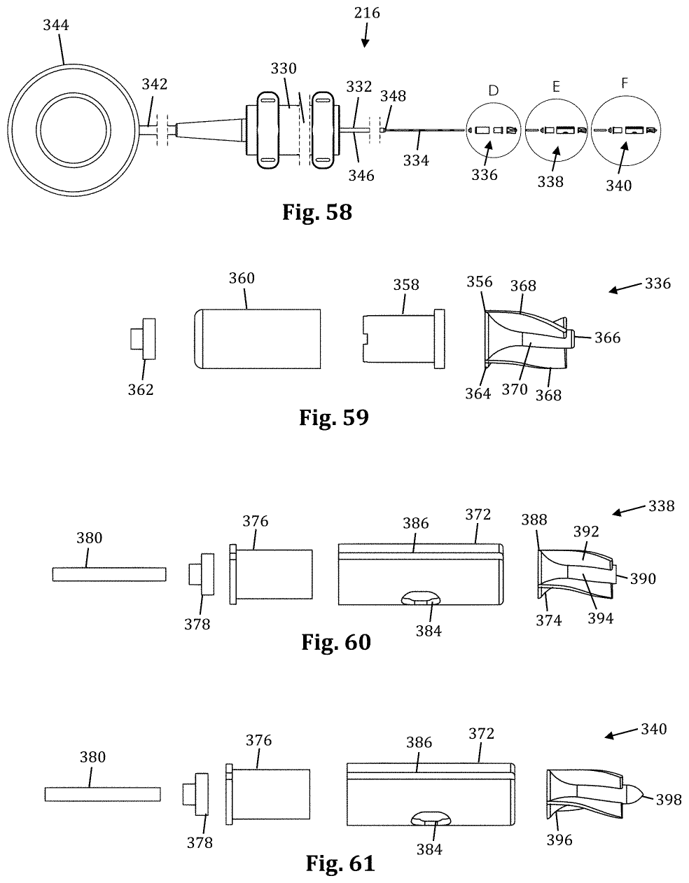

FIGS. 58-67 illustrate the pump subsystem 216 in greater detail according to one embodiment. Continued reference to FIGS. 43-45 may be made to understand how the various components of the sheath 212, catheter 214, and pump subsystem 216 interact with one another. By way of example only, the pump subsystem 216 comprises a motor assembly 300, a drive cable assembly 332, a drive shaft, and a plurality of impeller pump assemblies arranged in a linear or tandem fashion, for example a first or proximal pump assembly 336, a second or middle pump assembly 338, and a third or distal pump assembly 340. For the purpose of illustration, the embodiment described herein by way of example includes three pump assemblies, however it should be understood that the number of pump assemblies employed is scalable depending upon the specific needs of the patient, so long as there is a minimum of two impeller pumps present (e.g. a first or proximal pump assembly 336 and a second or distal pump assembly 340). By way of example, the motor assembly 330 includes an electric motor, a drive cable assembly coupler, and a purge tubing manifold, and is configured to transmit rotational energy to the and purge fluid to the drive cable assembly 332. The motor assembly 330 is connected to a control unit (not shown) by way of a cable 342 and cable connector 344. The cable 342 is an electrical and hydraulic cord that conducts electrical power from the control unit (via cable connector 344) to the motor assembly 330, and transmits purge fluid to/from the cable connector 344 to/from the motor assembly 330.

By way of example, the drive cable assembly 332 may be a flexible torque cable having an outer drive sheath 346 and an inner drive sheath 348. The drive cable assembly 332 may be configured to transmit rotational energy to the drive shaft 334 and purge fluid power to the proximal pump assembly 336. The drive shaft 334 may be a hollow shaft having rigid segments 350, flexible segments 352, and tension spring segments 354. For example, the rigid segments 350 support pump impellers, the flexible segments 352 allow flex between impellers for insertion into patient anatomy, and tension spring segments 354 provide axial compression force for hydrodynamic bearings. The drive shaft 334 may also provide a conduit for purge fluid from the proximal pump assembly 336 to the middle pump assembly 338 and/or the distal pump assembly 340.

An example of the first or proximal pump assembly 336 will now be described with particular reference to FIGS. 59, 65, and 70. By way of example, the proximal pump assembly 336 includes an impeller 356, a bearing 358, a bearing housing 360, and a drive shaft collar 362. The proximal pump assembly 336 does not have a housing or shroud in this example embodiment because upon insertion the proximal pump assembly 336 is positioned within the proximal shroud 270 of the catheter 214 such that the impeller 356 aligns with the flow ports 298 of the shroud 270 to enable blood flow out of the proximal pump assembly 336 (or into the proximal pump assembly 336 depending upon the flow direction). The impeller 356 has a proximal base 364, a distal end 338, and a plurality of blades 368 (e.g. straight or curved) extending along the hub 370 from the base 364 to the distal end 366. The impeller 356 further includes an axial lumen extending therethrough configured to receive the drive shaft 334 therein, thereby coupling the drive shaft 334 to the impeller 356 so that the drive shaft 334 may transfer rotational energy from the motor assembly 330 to the proximal pump impeller 356 to draw blood flow through the proximal pump assembly 336.

The bearing 358 is positioned proximal of the impeller 356 and comprises a generally cylindrical rotary hydrodynamic shaft bushing, that constrains the drive shaft for rotational axial alignment. The bearing 358 also transmits purge fluid to the proximal impeller 356. The bearing housing 360 is a generally cylindrical rigid tubular member configured to contain the proximal bearing 336 therein. The drive shaft collar 362 is positioned proximal of the bearing 358 and comprises a rigid element attached to the drive shaft 334. The drive shaft collar 362 reacts the axial tension spring force from the drive shaft 334 on the proximal end of the bearing 358 creating a hydrodynamic seal with the bearing 358.

An example of the second or middle pump assembly 338 will now be described with particular reference to FIGS. 60, 64, and 70. By way of example, the middle pump assembly 338 includes a shroud 372, an impeller 374, a bearing 376, a drive shaft collar 378, and a drive shaft sleeve 380. The shroud 372 in this case is necessary because the middle pump assembly 338 is positioned within the lumen 314 of the expandable cannula 272. By way of example only, the shroud 372 is generally cylindrical and includes an inner cavity 382, at least one flow port 384, and an alignment feature 386. The inner cavity is sized and configured to contain the impeller 374 and a substantial portion of the bearing 376 therein. The at least one flow port 384 is configured to align with a flow port 316 in the cannula 272 to allow the fluid to flow into or out of the middle pump assembly 338 (depending on flow direction). The alignment feature 386 is configured to interact with a corresponding feature on the cannula 272 described above. The alignment feature 386 of the present example comprises an elongated axially-oriented groove or track configured to slidably mate with a complementary alignment feature in the cannula 272 (e.g. a corresponding axially-aligned tongue or rail in the inner lumen 314 described above) to ensure rotational alignment of the flow ports 316, 384.

The impeller 374 has a proximal base 388, a distal end 390, and a plurality of blades 392 (e.g. straight or curved) extending along the hub 394 from the base 388 to the distal end 390. The impeller 374 further includes an axial lumen extending therethrough configured to receive the drive shaft 334 therein, thereby coupling the drive shaft 334 to the impeller 374 so that the drive shaft 334 may transfer rotational energy from the motor assembly 330 to the proximal pump impeller 374 to draw blood flow through the middle pump assembly 338.

The bearing 376 is positioned proximal of the impeller 374 and comprises a generally cylindrical rotary hydrodynamic shaft bushing, that constrains the drive shaft 334 for rotational axial alignment. The bearing 376 also transmits purge fluid to the middle impeller 374. The drive shaft collar 378 is positioned proximal of the bearing 376 and comprises a rigid element attached to the drive shaft 334. The drive shaft collar 378 reacts the axial tension spring force from the drive shaft 334 on the proximal end of the bearing 376 creating a hydrodynamic seal with the bearing 376. The drive shaft sleeve 380 by way of example only is a flexible tube attached and sealed to the drive shaft 334 to constrain purge fluid within the drive shaft 332.

An example of the third or distal pump assembly 340 will now be described with particular reference to FIGS. 61, 66, 67, and 70. By way of example, the distal pump assembly 340 includes a shroud 372, an impeller 396, a bearing 376, a drive shaft collar 378, and a drive shaft sleeve 380. The distal pump assembly 340 is substantially similar to the middle pump assembly 338 described above, and in fact several components including the shroud 372, bearing 376, drive shaft collar 378, and drive shaft sleeve 380 identical in form and function, and a repeat discussion is not necessary. The impeller 374 has a proximal base 388, a distal end 390, and a plurality of blades 392 (e.g. straight or curved) extending along the hub 394 from the base 388 to the distal end 390. The impeller 374 further includes an axial lumen extending therethrough configured to receive the drive shaft 334 therein, thereby coupling the drive shaft 334 to the impeller 374 so that the drive shaft 334 may transfer rotational energy from the motor assembly 330 to the proximal pump impeller 374 to draw blood flow through the middle pump assembly 338. The distal impeller 396 differs from the middle impeller 374 in that the distal impeller 396 also includes a distal cap 398 configured to seal the distal end of the axial lumen.

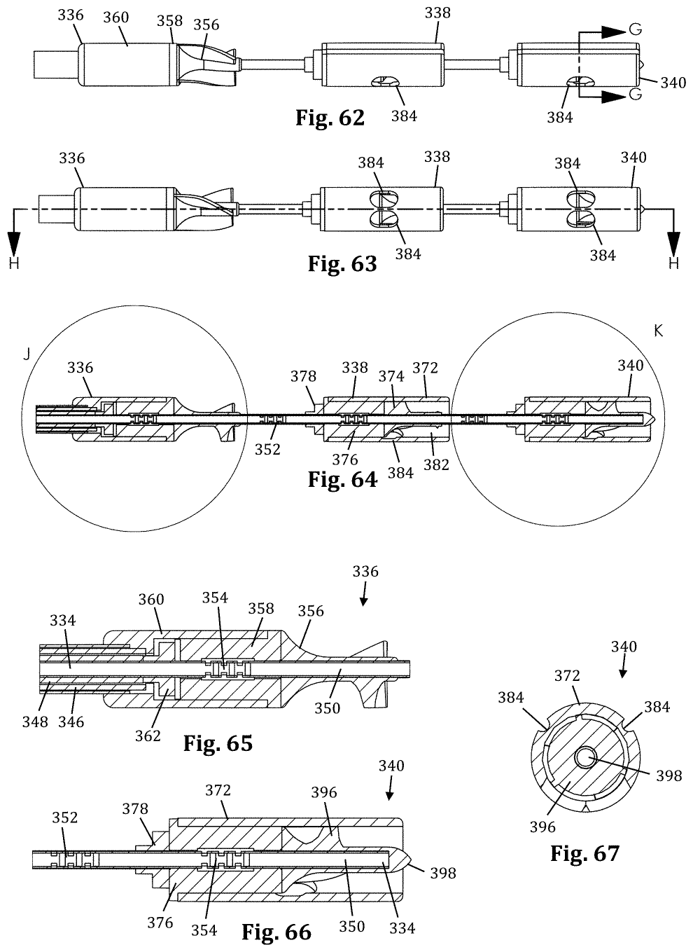

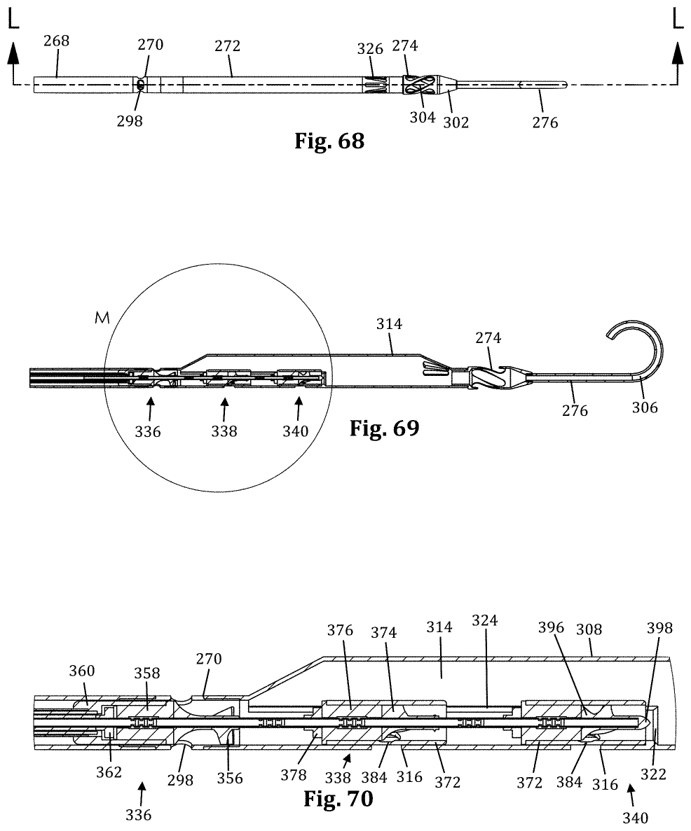

FIGS. 68-70 illustrate by way of example only the positioning of the pump assemblies 336, 338, 340 within the catheter 214 according to one embodiment, and as described above.

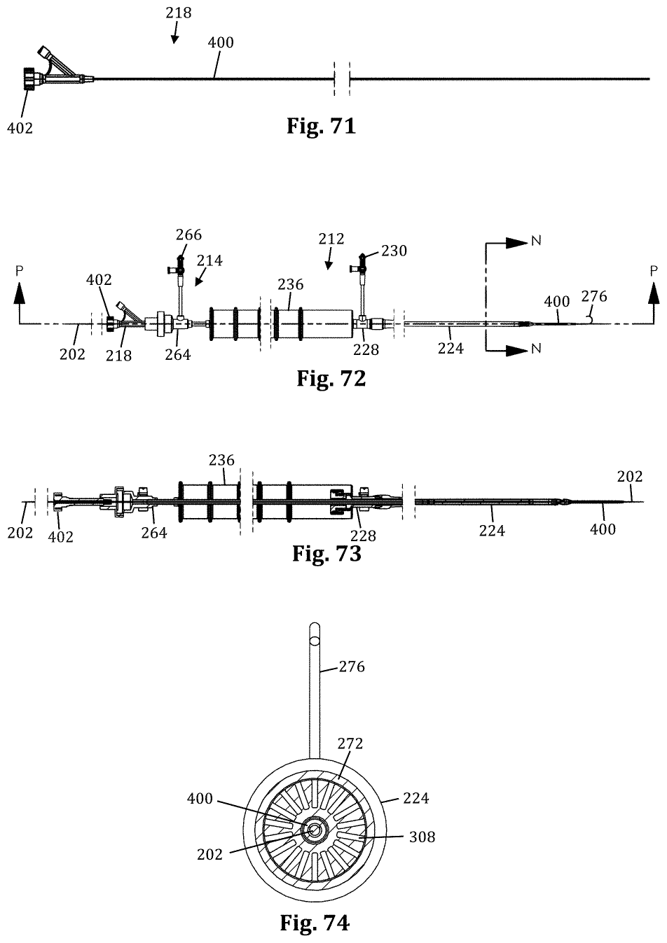

FIG. 71 illustrates an example of an obturator 218 according to one embodiment. By way of example, the obturator of the present example includes a flexible tube 400 configured to receive a guide wire therein, and a guide wire hemostasis valve 402 configured to seal blood inside the patient and allow for axial translation of the catheter 214 over the guide wire 202.

FIGS. 72-74 illustrate the percutaneous blood pump assembly 210 configured for insertion into a patient according to one embodiment. By way of example only, and as shown in FIG. 72, the obturator 218 is inserted into the catheter 214, which in turn is inserted into the sheath 212. As shown in FIG. 73, a guide wire 202 may be inserted through the obturator 218. As shown in FIG. 74, the self-expanding cannula 272 is held in a collapsed state for insertion by the sheath shaft 224. Notably, the expandable body 308 in the collapsed state occupies space that will be occupied by one or more pump assemblies (e.g. second pump assembly 338 and/or third pump assembly 340, and so on) upon expansion of the expandable cannula 272. This enables a cannula 272 with a smaller (collapsed) diameter to be inserted through the body, improving the ease of access.

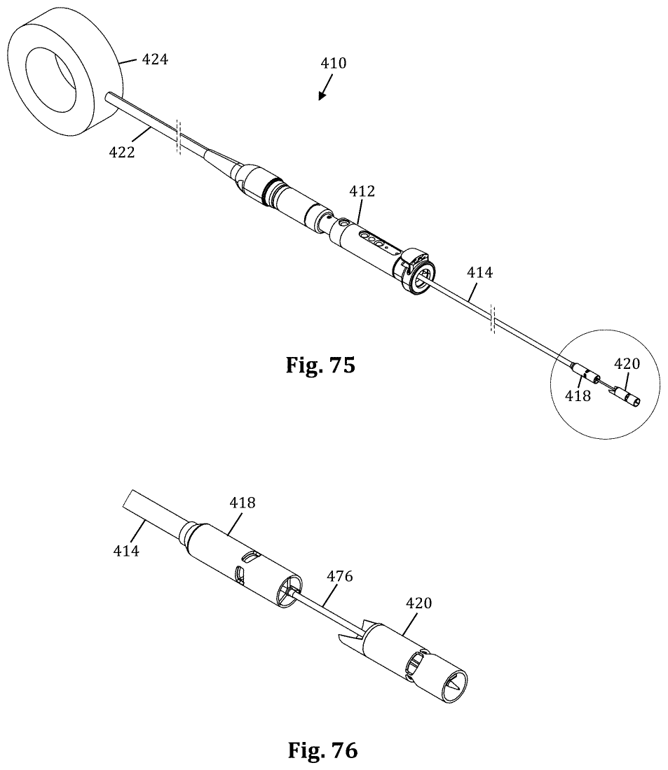

FIGS. 75-95 illustrate an example of a pump subsystem 410 configured for use with the blood pump system 10 disclosed herein above according to one embodiment of the disclosure. By way of example only, the pump subsystem 410 comprises a motor assembly 412, a drive cable assembly 414, a drive cable 416 (similar to drive cable 32), and a plurality of impeller pump assemblies arranged in a linear or tandem fashion, for example a first or proximal pump assembly 418 and a second or distal pump assembly 420. For the purpose of illustration, the embodiment described herein by way of example includes two pump assemblies, however it should be understood that the number of pump assemblies employed is scalable depending upon the specific needs of the patient, so long as there is a minimum of two impeller pumps present (e.g. a first or proximal pump assembly 418 and a second or distal pump assembly 420). By way of example, the motor assembly 412 includes an electric motor, a drive cable 416 coupler, and a purge tubing/drive sheath manifold, and is configured to transmit rotational energy to the drive cable 416 and purge fluid to/from the drive cable assembly 414. The motor assembly 412 is connected to a control unit (not shown) by way of a cable 422 and cable connector 424. The cable 422 is an electrical and hydraulic cord that conducts electrical power from the control unit (via cable connector 424) to the motor assembly 412, and transmits purge fluid to/from the cable connector 424 and to/from the motor assembly 412.

By way of example, the drive cable assembly 414 may include a drive cable 416, an outer drive sheath 426, and an inner drive sheath 428. The drive cable assembly 414 may be configured to transmit rotational energy to the drive cable 416 and purge fluid pressure and flow to the proximal pump assembly 418 for operation of hydrodynamic bearings. Fresh purge fluid is transmitted to the proximal pump assembly 418 via the outer drive sheath 426 which is coaxially arranged outside the inner drive sheath 428. The inner drive sheath 428 houses the drive cable 416 and waste purge fluid that flushes the wear particles outside the patient. The drive cable 416 is made from multiple wires (filars) and layers for torque transmission and flexibility suitable for the anatomical route required. The drive cable assembly 414 is connected to the bearing assembly 438 by way of a sheath adapter 580 and cable adapter 486. By way of example only, the sheath adapter 580 includes a distal post 582 sized and configured to nest within the inner lumen 488 of the bearing housing 478, and may be secured to the bearing housing 478 by any suitable mechanism (e.g. threaded connection, adhesives, etc.). The sheath adapter 580 has an inner lumen 584 configured to bond the outer drive sheath 426 and seat the inner drive sheath 428. The inner lumen 584 has axial grooves 586 formed therein to allow for the passage of purge fluid from the outer sheath to the proximal pump assembly.

FIGS. 77-87 illustrate an example of a first or proximal pump assembly 418 according to one example embodiment. By way of example, the first or proximal pump assembly 418 includes an impeller assembly 436 and a bearing assembly 438 each contained with in a housing 440. The housing 440 of the instant example comprises a generally cylindrical tubular member having an inner lumen 442 sized and configured to contain the impeller assembly 436 and the bearing assembly 438 therein. The housing 440 further comprises a plurality of flow apertures 444 configured to align with the impeller 448 upon assembly to facilitate ingress into or egress from the inner lumen 442 (for example depending on the flow direction) and a plurality of axial slots 446 in the inner lumen wall at the distal end of the housing 440. Each axial slot 446 is configured to receive one radial support strut 468 of the tip bearing 450. Notably, in the instant example embodiment the proximal pump housing 440 is a part of the first pump assembly 418 and not catheter assembly (for example as described above), and as such both the proximal and distal pump assemblies 418, 420 have attached pump housings and impellers of the same or closely similar diameter.

The impeller assembly 436 includes a proximal pump impeller 448, a tip bearing 450, an drive shaft 452, and a collar 454. The proximal pump impeller 448 has a proximal base 456, impeller fulcrum 458, a plurality of blades 460 (e.g. straight or curved) extending along the hub 462 from the base 456 to the impeller fulcrum 458, and a proximal shaft 464 extending proximally from the base 456 and configured to engage the bearing assembly 438 as described below. The proximal pump impeller 448 further includes an axial lumen extending proximally therethrough configured to receive the cable adapter 486 therein, thereby coupling the drive cable 416 to the proximal pump impeller 448 so that drive cable 416 may transfer rotational energy from the motor assembly to the proximal pump assembly 418. The proximal pump impeller 448 further includes an axial lumen extending distally therethrough configured to receive the drive shaft 452 therein, thereby coupling the drive shaft 452 to the proximal pump impeller 448 so that the drive shaft 452 may transfer rotational energy from proximal pump impeller 448 to the distal pump assembly 420.

The tip bearing 450 has a base 466, a plurality of radial struts 468, and a central aperture 470 extending axially through the base. The radial struts 468 extend radially outward from the base 466 and are sized to span the distance between the base 466 and the axial slots 446 of the housing 440 so that the tip bearing 450 may be relatively constrained within the axial slots 446. The radial struts 468 may be straight or curved to form an inducer to precondition the fluid flow path to minimize hydraulic instability (e.g. flow separation, cavitation, vortices). When radial struts 468 are curved to form an inducer, the outer ends are configured straight for axial alignment with slots 446. The central aperture 470 is sized and configured to rotatably receive the drive shaft 416 therethrough and allow the drive shaft 416 and therefore the proximal pump impeller 448 to rotate at high speed while maintaining axial alignment of the proximal pump impeller 448 to ensure coaxial rotation. Although shown in FIG. 78 by way of example only as having three radial struts 468, the tip bearing 450 may have any number of radial struts 468 without departing from the scope of the disclosure.

The tip bearing 450 of the present example is positioned centrally in the pump housing 440 to align the impeller distal end or fulcrum 458 to centerline and allow torque transmission from the proximal pump assembly 418 to the distal pump assembly 420 without deflection of the proximal pump impeller 448 which may cause the tips of the impeller blades 460 to rub against the pump housing 440. The tip bearing 450 is self-aligning in an axial direction due to the axial slots 446 of the housing 440 having longer lengths than the axial length of each radial support strut 468. This allows hydrodynamic bearings on both ends (proximal and distal) of the proximal pump impeller 448 to function without negative effect from component axial manufacturing tolerance stack up.

By way of example, the drive shaft 452 comprises a hollow shaft with tension spring segments 472 for loading hydrodynamic bearings (e.g. tip bearing 450 and distal pump 420) and a middle flexible segment 474 for bending during pump insertion through torturous anatomy. The drive shaft 452 may be sealed with a flexible jacket or drive shaft cover 476 for transporting the purge fluid to one or more distal pump assemblies 420. The drive shaft 452 may be constructed of a single piece (e.g. laser cut hypo tube) or of multiple pieces (e.g. solid hollow shaft for rigid segments, flexible drive cable for middle flexible segments 474, and laser cut thin-wall tube or single-wire coiled tension spring for tension spring segments 472, or any combination therein).

The collar 454 is attached to the drive shaft 452 distal to the tip bearing 450. The collar 454 puts the tension spring segment 472 of coupling drive shaft under tensile load when attached (e.g. laser welded), reacting load to impeller fulcrum 458. This squeezes the tip bearing 450 ends for hydrodynamic effect whereby thin film of pressurized purge fluid (e.g. saline solution, dextrose solution) leaks out of rotating interface at end faces (e.g. proximal and distal) of tip bearing 450 resulting in a “hydroplaning” effect that minimizes the temperature increase from rotational friction while maintaining axial alignment of the proximal pump impeller 448 and impeller housing 440. Excessive heat from rotational friction is known to activate the clotting cascade which poses risk of vascular embolism to the patient. Excessive impeller runout can cause flow disturbances within the impeller flow region reducing pump efficiency, cause blood damage or activate platelets.

The bearing assembly 438 of the instant example embodiment includes a bearing housing 478, distal bushing 480, proximal bushing 482, compression spring 484, and threaded cable adapter 486. By way of example, the bearing housing 478 comprises a generally cylindrical tubular member having an inner lumen 488 sized and configured to house the distal bushing 480, proximal bushing 482, compression spring 484, threaded cable adaptor 486, and impeller proximal shaft 464 therein, and has a smooth outer surface 490 configured for attachment to the housing inner lumen 442. The distal bushing 480 is fixed to the bearing housing 478 and includes axial grooves 492 on an inner diameter to transport purge fluid along the impeller shaft 464 to a proximal-facing hydrodynamic bearing surface 494 at the impeller base 456. Alternatively, bearing housing 478 may be integrated into impeller housing 440. Alternatively, bearing housing 478 and distal bushing 480 may be integrated into impeller housing 440. The compression spring 484 applies force to the proximal bushing 482 that is slip-fit to the bearing housing 478 in an axial “floating” manner. The proximal bushing 482 has axial grooves 496 on an inner diameter for purge flow, and proximal grooves 498 on a proximal face for purge flow from sheath (not shown but see description above) into the proximal pump assembly 418.

The threaded cable adapter 486 has a proximal flange 500, and a distal-extending post 502. The proximal flange 500 reacts the force that the compression spring 484 applies to the proximal bushing 482. The distal-extending post 502 has a distal threaded coupler 504 and an inner cavity 506 sized and configured to receive at least a portion of the drive cable 416 therein. The inner cavity 506 also includes a thin-wall crimping element 508 configured to crimp the drive cable 416 onto a pin mandrel 510 inside distal end of drive cable 416 to securely connect the cable adapter 486 to the drive cable 416.

The proximal impeller shaft 464 and cable adapter 486 may have side-holes 512 formed therein to allow purge flow into the central lumens of the cable adapter 486, proximal impeller 448, and drive shaft 452 to supply purge fluid to the distal pump(s) 420.

After crimp connection of the drive cable 416 to the cable adapter 486 (e.g. by way of pin mandrel 510), the drive cable 416 is essentially threaded to impeller shaft 464 (e.g. by way of a threaded engagement between the threaded coupler 504 of the cable adaptor 486 and a threaded cavity 514 of the impeller shaft 464. The proximal bushing 482, distal bushing 480, and bearing housing 478 fitted to compress the compression spring 484, connected to proximal pump impeller 448 and proximal pump housing 440 form the proximal pump assembly 418.

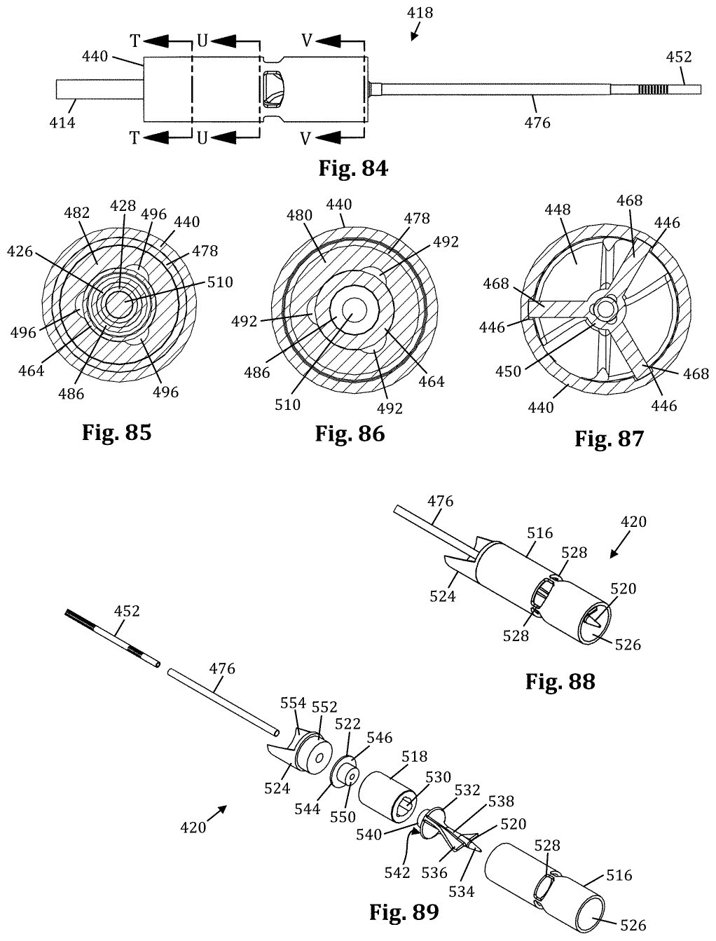

By way of example, FIG. 85 provides an axial cross-section view of the proximal pump assembly 418 with the section cut along line T-T of FIG. 84 (e.g. through the proximal bushing 482). FIG. 86 provides an axial cross-section view of the proximal pump assembly 418 with the section cut along line U-U of FIG. 84 (e.g. through the distal bushing 480). FIG. 87 provides an axial cross-section view of the proximal pump assembly 418 with the section cut taken along line V-V of FIG. 84 (e.g. through the tip bearing 450).

FIGS. 88-95 illustrate an example of a second or distal pump assembly 420 according to one example embodiment. By way of example, the distal pump assembly 420 includes a housing 516, bushing 518, impeller 524, proximal collar 522, and a proximal end cap 524. The housing 516 of the instant example comprises a generally cylindrical tubular member having an inner lumen 526 sized and configured to contain the various components described herein. The housing 516 further comprises a plurality of flow apertures 528 configured to align with the impeller 520 upon assembly to facilitate ingress into or egress from the inner lumen 526 (for example depending on the flow direction). The bushing 518 is press-fit or bonded into the inner lumen 526 of the housing 516 and includes axial grooves 530 on an inner diameter to transport purge fluid to the proximal-facing hydrodynamic bearing surface 542 at the impeller base 532 and distal-facing hydrodynamic bearing surface 546 on the base 544 of the proximal collar 522. Alternatively, bushing 518 may be integrated into housing 516.

The impeller 520 has a proximal base 532, a distal end 534, a plurality of blades 536 (e.g. straight or curved) extending along the hub 538 from the base 532 to the distal end 534, and a proximal shaft 540 extending proximally from the base 532 and configured to engage the bushing 518. The impeller 520 further includes a proximal-facing hydrodynamic bearing surface 542 configured to hydrodynamically engage a distal-facing outer surface 544 of the bushing 518, and an axial lumen extending therethrough configured to receive the drive shaft 452 therein, thereby coupling the drive cable 416 to the impeller 520 (by way of drive shaft 452, proximal pump impeller 448, and threaded cable adapter 486 as described above so that the drive shaft 416 may transfer rotational energy from the motor assembly 412 to the distal pump impeller 520 to draw blood flow through the distal pump assembly 420.

By way of example, the distal pump assembly 420 is shown with a hydrodynamic bearing arrangement similar to the tip bearing 520 of the proximal pump assembly 418 described above, where the drive shaft tension spring segment 472 is stretched during assembly and fixed by the attachment (e.g. by welding) to the proximal collar 522. The proximal collar 522 includes a generally cylindrical base 544 having a planar distal-facing hydrodynamic bearing surface 546, and a distal shaft 550 having an inner lumen extending therethrough. The distal shaft 550 is sized and configured to be received within the inner lumen of the bushing 518 while the outer diameter of the base 544 is sized and configured for rotational clearance with the inner lumen 526 of the housing 516. The proximal end cap 524 generally cylindrical distal shaft 552 sized and configured for press-fit or bonding into the housing 516. The proximal end cap 524 may have a shaped proximal end 554 having a generally concave surface (for example) shaped to fill blood stasis volume outside the high velocity flow streams to prevent thrombus formation. Alternatively, at least one radial blade (not shown) may be attached to the outer surface of the drive shaft cover 476 near the end cap 524 to induce turbulence that washes the volume and prevents fluid stasis.

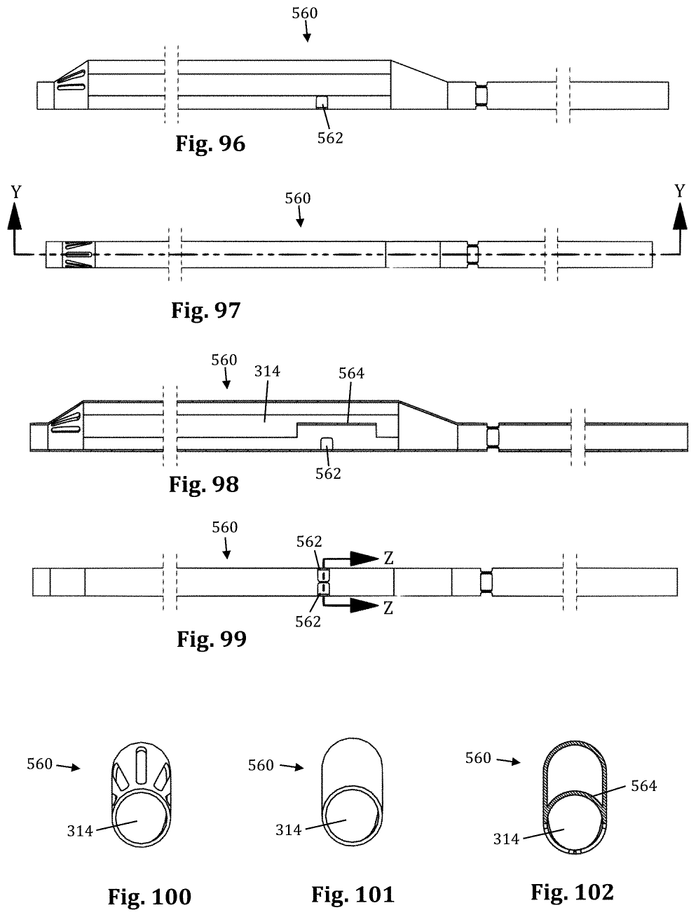

FIGS. 96-102 illustrate an example of an expandable cannula 560 forming part of the percutaneous blood pump system 210, according to one embodiment. The cannula 560 of the present example comprises a thin-wall, self-expanding, re-collapsible, cylindrical tube providing a conduit for blood flow out of heart (for example). The cannula 560 is inserted in collapsed configuration (see, e.g. FIG. 74) and expands to an expanded operating configuration upon emergence from the sheath 212. The expanded cross-section is configured to be suitable for the desired length of the cannula 560, number of tandem pumps operating, and desired maximum pressure drop from the cannula inlet to the pump inlets at the desired maximum system flow rate. The cannula 560 of the present example is substantially similar to the cannula 272 described above such that description of like features will not be repeated, and those features that are the same as above will be referenced with the same numbers used above, however features that are new or different will be assigned new reference numbers and described accordingly.

By way of example only, the expandable cannula 560 comprises a single inner lumen 314, and one or more flow port(s) 562 formed in the body 308 (by way of example). Unlike the flow ports 316 on the cannula 272 above, the flow ports 562 of the instant example may be formed not only on the “bottom” of the cannula 560 but also partially on the lateral sides. The reason for this is that the cannula 560 has an alignment feature in the form of a tubular pump guide 564. By way of example, the tubular pump guide 564 may be a form-fitting cover that blocks flow from any ports that may be facing the tubular pump guide 564 upon insertion of the pumps such as by way of example distal proximal or distal pumps 418, 420 of shown in FIG. 76 into the cannula 560. Thus, the tubular pump guide 564 enables the use of a distal pump shroud with a full 360° array of ports so a user does not have to align rotationally to ports 562 in the cannula 560.

The cannula 560 of the present example is configured for use with a proximal pump housing as part of the pump assembly (for example like the proximal pump assembly 418 described above) instead of having the housing part of the catheter (for example like the proximal pump assembly 336 described above). The cannula 560 and proximal guide shaft may be all one piece back to the hemostasis valve, or of two or more pieces, for example proximal and middle with unobstructed 360° ports, and a distal expandable segment as described above.

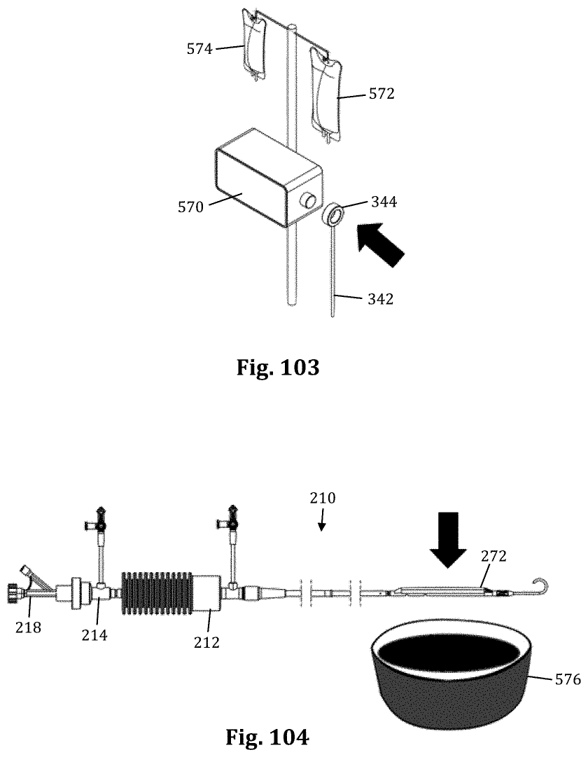

FIGS. 103-115 illustrate a method of using the percutaneous blood pump system 210 described above, according to one example embodiment. The first step is to prime the system 210 for use. To accomplish this, as shown in FIG. 103, a non-sterile technician may connect the cable connector 344 to a control console 570, and also set up the control console 570 with a purge fluid bag 572 and purge fluid waste bag 574. The technician then activates the console 570 to prime the drive cable assembly 332 with purge fluid (e.g. heparnized 5% dextrose solution).

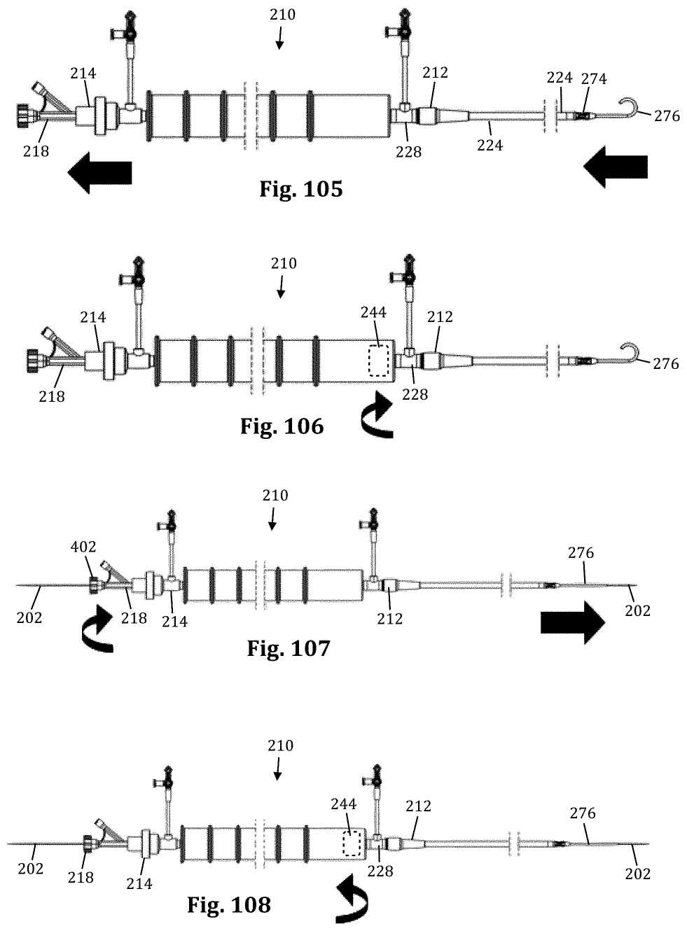

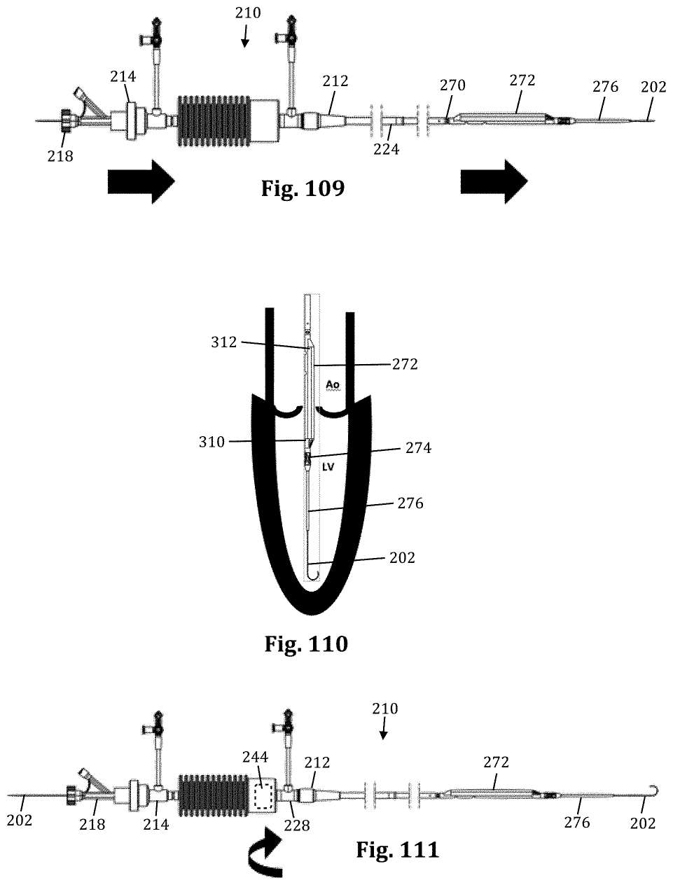

The next step is to establish femoral artery access and track the guide wire 202 into the left ventricle of the heart. At this point the blood pump system 210 is configured for initial insertion, namely the obturator 218 is inserted into the catheter 214, which is inserted into the sheath 212. The user first hydrates the lubricious coating of the self-expanding cannula 272 in a bowl of sterile saline 576 (e.g. FIG. 104). The user may then sheathes the self-expanding cannula 272 by holding the sheath 212 and pulling the catheter 214 until the tip housing 274 is seated against the distal end of the sheath shaft 224 (e.g. FIG. 105). The next step is to secure the sheath hemostasis valve 228 onto the catheter 214 by rotating the valve handle 244 (e.g. FIG. 106). Next, the user may insert the guide wire 202 by backloading the guide wire 202 through the atraumatic tip 276 of the catheter 214 until the guide wire 202 emerges from the obturator 218 at the proximal end. The user may then close the obturator guide wire hemostatis valve 402 over the guide wire (e.g. FIG. 107). The user may then track the sheath 212 and catheter 214 over the guide wire 202 into the descending aorta. The user then loosens the sheath hemostasis valve 228 by rotation the valve handle 244 in the opposite direction (e.g. FIG. 108). The user then unsheathes the self-expanding cannula 272 by holding the sheath 212 and pushing the catheter 214 until the proximal shroud 270 is distal of the distal end of the sheath shaft 224 (e.g. FIG. 109). Expanding the cannula 272 opens the space to be occupied by one or more of the pump assemblies of the pump subsystem. The user then tracks the catheter tip housing 274 over the guide wire 202 and into the left ventricle. At this point the cannula 272 is seated in the desired intra-valvular position (e.g. with the distal end 310 of the cannula 272 positioned in the left ventricle and the proximal end 312 of the cannula 272 positioned in the aorta, as shown in FIG. 110).

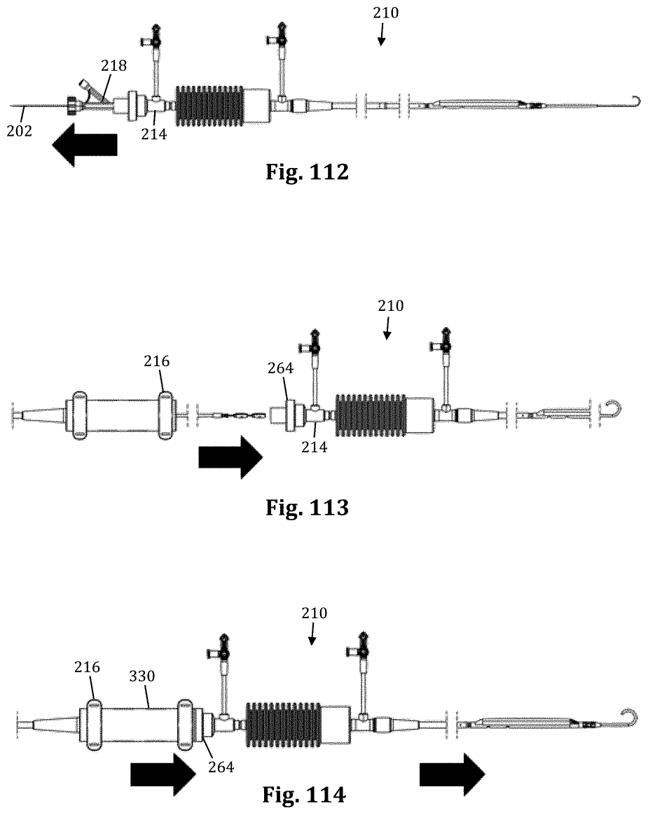

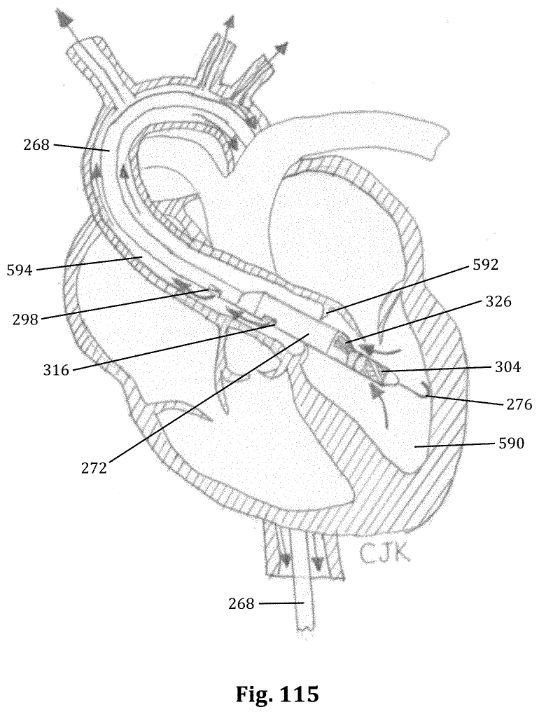

To insert the pump system 216, the user must first remove the guide wire 202 and obturator 218 from the catheter 214. To accomplish this, the user secures the sheath hemostasis valve 228 onto the catheter by rotating the valve handle 244 (e.g. FIG. 111). The user may then remove the obturator 218 and guide wire 202 by pulling each proximally from the catheter 214 (e.g. FIG. 112). The pump subsystem 216 is then introduced by inserting through the catheter hemostasis valve 264 (e.g. FIG. 113). The user then tracks the pump subsystem 216 into the catheter 214 until the pump motor assembly 330 connects with the catheter hemostasis valve 264, clicking to secure (e.g. FIG. 114). At this point, the proximal pump assembly will be located in the catheter immediately proximal of the cannula 272, and the distal pump assembly will be located inside the cannula in the space previously occupied by the collapsed cannula prior to expansion. The user may then verify the catheter position and suture the sheath to the patient. By way of example only, the percutaneous blood pump system 210 may now be used to pump blood from the left ventricle 590 of the heart, across the aortic valve 592 and into the aorta 594, as shown in FIG. 115.

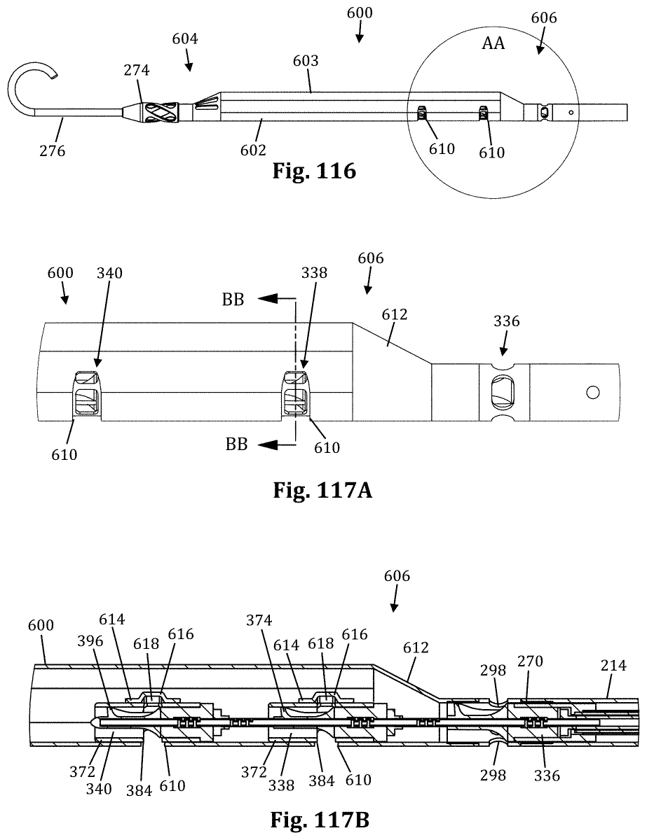

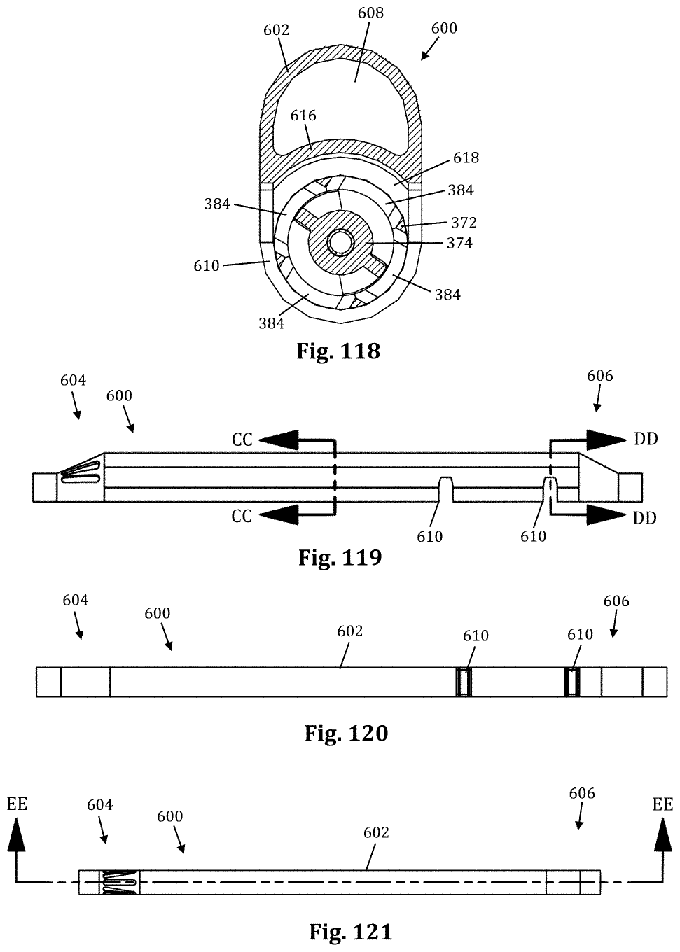

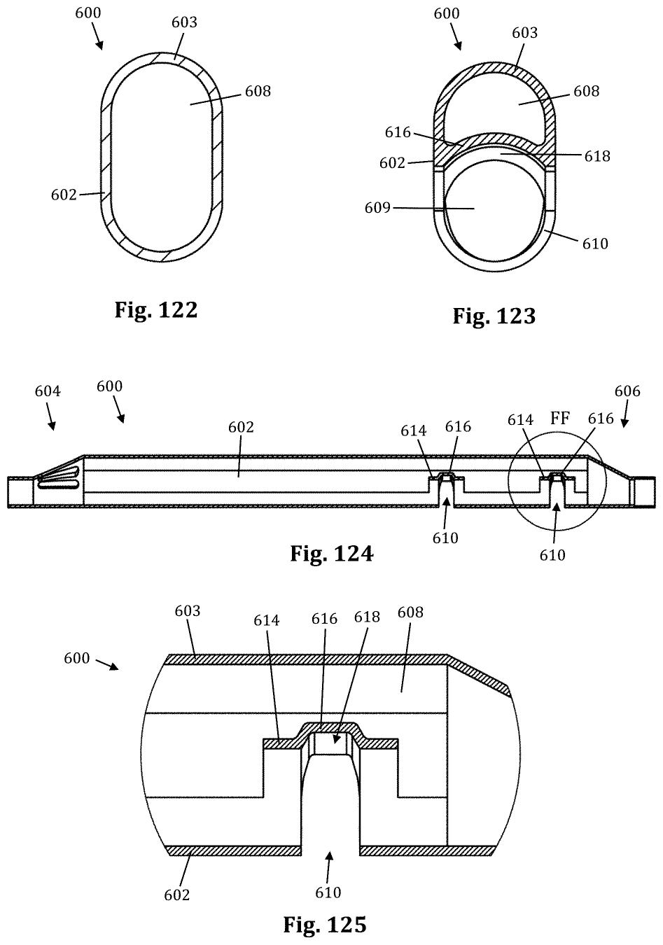

FIGS. 116-125 illustrate another example of an expandable cannula 600 forming part of the percutaneous blood pump system 210 described above, according to some embodiments. The cannula 600 of the present example comprises a thin-wall, self-expanding, re-collapsible, cylindrical tube providing a conduit for fluid flow (e.g. blood flow out of the heart). By way of example, the cannula 600 may be inserted in a collapsed configuration (see, e.g., the collapsed configuration of cannula 272 shown in FIG. 74) and expanded to an expanded operating configuration upon emergence from the sheath 212 as described above. The expanded portion 603 may be configured to be suitable for the desired length of the cannula 600, number of tandem pumps operating, and desired maximum pressure drop from the cannula inlet to the pump inlets at the desired maximum system flow rate. The cannula 600 of the present example is substantially similar to the cannula 272 described above such that description of like features will not be repeated, and those features that are the same as above will be referenced with the same numbers used above, however features that are new or different will be assigned new reference numbers and described accordingly.

By way of example only, the expandable cannula 600 comprises an expandable body 602 including an expanded portion 603, a distal end 604, a proximal end 606, inner lumen 608, and one or more flow port(s) 610 formed in the bottom portion of the body 602 (by way of example). The expandable body 602 comprises a thin-walled, self-expanding, re-collapsible tube made from flexible polymer and reinforcing frame, and includes an inner lumen 608 extending axially therethrough and a proximal taper 612 at the proximal end 606 of the expandable body 602. The inner lumen 608 is sized and configured to allow passage of a number of instruments and components therethrough, including but not limited to the obturator 218, guide wire 202, and the like, and is also configured to allow the flow of fluids therethrough.

The inner lumen 608 is also configured to house one or more middle and/or distal pump assemblies 338, 340 that are inserted into the cannula 600 after expansion of the expandable body 602. A flow port 610 is provided for each middle and/or distal pump assembly 338, 340 within the cannula 600 to allow the fluid to flow through tandem arranged impellers (e.g. impellers 374, 396 above) in parallel through the cannula 600. To ensure that the pump assemblies are properly aligned with the flow ports 610 upon insertion into the cannula 600, the inner lumen may further include one or more pump alignment or location features, including but not limited to (and by way of example only) a close fitting sleeve 614 positioned about the flow ports 610 and configured to flushly receive the middle and/or distal pump assemblies 338, 340 therein and position the pump assemblies such that the impellers (e.g. impellers 374, 396 above) are aligned with the flow ports 610. In some embodiments, the cannula 600 may include one or more other pump alignment/location features previously described, such as a laterally-oriented pump stop and/or an anti-rotation feature. By way of example only, the pump stop may be a physical barrier to prevent advancement of the pump assemblies once the shroud flow ports 384 are laterally aligned with the cannula flow ports 610. In some embodiments, the anti-rotation feature may comprise a spine, snap-fit, and/or an elongated axially-oriented tongue or rail in the inner lumen 608 configured to slidably mate with a complementary alignment feature (e.g. a corresponding axially-aligned groove or track) formed on the outer surface of the middle and/or distal pump shroud(s) 372 to prevent rotation of the shroud(s) 372 to ensure rotational alignment of the flow ports 384, 610 during operation of the pump.

In some embodiments, the cannula 600 may have an alignment feature in the form of a tubular pump guide or sleeve 614 positioned within the inner lumen 608 and forming a channel 609 configured to receive the middle and/or distal pump shrouds 372 (including the middle and/or distal pumps 338, 340) therein. By way of example, when the middle and/or distal pump shrouds 372 are inserted within the channel(s) 609, the tubular pump guide 614 may be a form-fitting cover that prevents outflow from the impellers 374 from entering the inner lumen 608, thereby ensuring that the outflow from the impellers 374 exits the cannula 600 through flow ports 610. Unlike the tubular pump guide 564 of cannula 560 above, the tubular pump guide 614 of the present example may include a self-expanding internal partition 616 that expands to create a void 618 between the pump shroud 372 and the internal partition 616 while fluidly separating the void 618 from the inner lumen 608. Thus, the tubular pump guide 614 enables the use of a middle and/or distal pump shroud 372 with a full 360° array of flow ports 384 that a user does not have to align rotationally to ports 610 in the cannula 600 because the void 618 enables fluid outflow through the flow ports 384 of the pump shroud 372 facing the internal partition 616 and directs the outflow through the flow ports 610 of the cannula 600. Additionally, the flow ports 610 of the instant example may be formed not only on the “bottom” of the cannula 600 but also partially on the lateral sides, increasing the flow outlet area and flow rate. This example configuration is advantageous because it improves the flow rates of the middle pumps 338 and distal pumps 340.

In some embodiments, the cannula body 602, location features, anti-rotation features, and/or internal partition 616 are elastically collapsible when pulled inside the delivery sheath and elastically expandable when pushed outside the delivery sheath. By of example only, the cannula body 602, location features, anti-rotation features, and/or internal partition 616 may be made of an elastic material (e.g., rubber, plastic, etc.) or a reinforced elastic material (e.g., nitinol, stainless steel, etc.). In some embodiments, the cannula body 602, location features, anti-rotation features, and/or internal partition 616 may be made of dual-wall elastic material (e.g., rubber, plastic, etc.) having an internal space or void configured to receive pressurized fluid (e.g., saline, blood compatible gas, etc.) to facilitate expansion and/or contraction of the feature for delivery and/or removal into or out of the patient through the delivery sheath.

The proximal taper 612 funnels the fluid (e.g. blood) flow within the inner lumen 608 to the inlet of the proximal pump 336 and facilitates collapsing of the expandable body 602 for removal from the body. More specifically, to remove the expandable cannula 600 from the body, a user exerts an axial force in the proximal direction to pull the catheter back through the sheath 212. As the proximal taper 612 encounters the tip funnel 258 of the sheath 212 (see e.g. FIG. 48), the proximal taper 612 translates the axial force applied to the cannula body 602 by the tip tube 256 (due to its rigidity) into inward radial force to collapse the expandable body 602 for removal through the sheath 212.

In some embodiments, the cannula 600 of the present example may be configured for use with a proximal pump housing or shroud 270 including flow ports 298 that is part of the catheter 214, for example like the proximal pump assembly 336 described above and shown in FIG. 117B. In some embodiments, the cannula 600 of the present example may be configured for use with a proximal pump housing that is part of the pump assembly (for example like the proximal pump assembly 418 described above). In some embodiments, the cannula 600 and proximal guide shaft may be formed as one piece back to the hemostasis valve, or alternatively may be formed of two or more pieces.

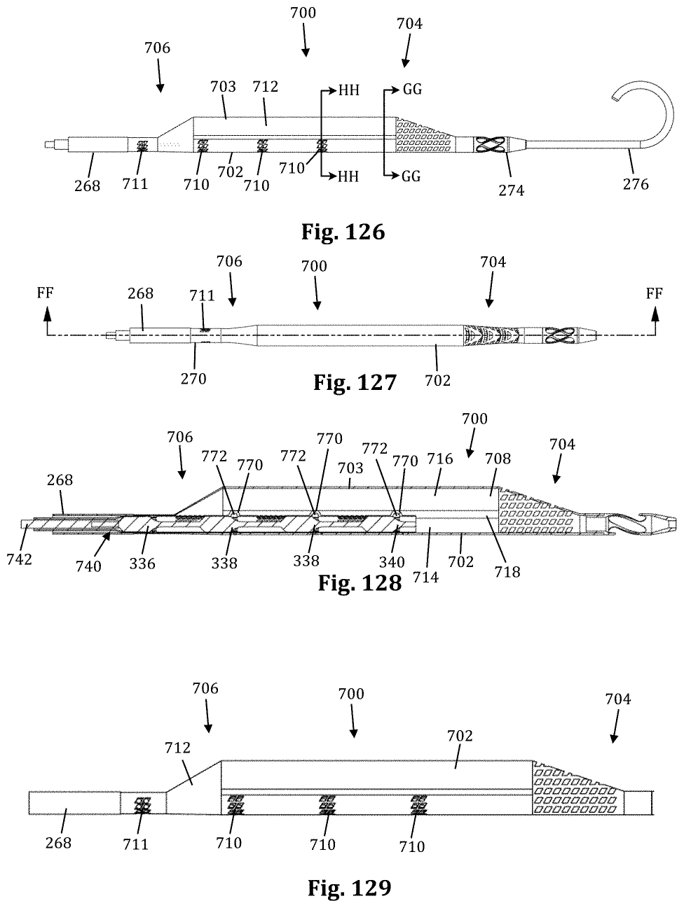

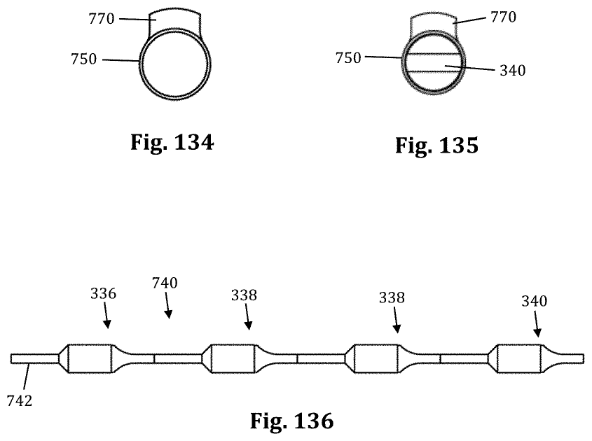

FIGS. 126-136 illustrate another example of a distal portion of a catheter-pump assembly forming part of the percutaneous blood pump system 210 described above, according to some embodiments. The catheter-pump assembly of the present example embodiment includes an expandable cannula 700 and a pump assembly 730, which includes a shroud 750 and a plurality of impeller pump assemblies arranged in a linear or tandem fashion, for example a first or proximal pump assembly 336, a second or middle pump assembly 338, and a third or distal pump assembly 340, which may be any of the pump assemblies described herein above (see, e.g., FIGS. 58-70 and accompanying disclosure). For the purpose of illustration, the embodiment described herein by way of example includes four pump assemblies (e.g. a proximal pump assembly 336, a pair of middle pump assemblies 338, and a distal pump assembly 340), however it should be understood that the number of pump assemblies employed is scalable depending upon the specific needs of the patient, so long as there is a minimum of two impeller pumps present (e.g. a first or proximal pump assembly 336 and a second or distal pump assembly 340). As will be described, the pump assemblies are positioned within the shroud 750, which is positioned within the cannula 700. By way of example, the cannula 700 of the present example comprises a thin-wall, self-expanding, re-collapsible, cylindrical tube providing a conduit for fluid flow (e.g. blood flow out of the heart). By way of example, the cannula 700 may be inserted in a collapsed configuration (see, e.g., the collapsed configuration of cannula 272 shown in FIG. 74) and expanded to an expanded operating configuration upon emergence from the sheath 212 as described above. The expanded portion 703 may be configured to be suitable for the desired length of the cannula 700, number of tandem pumps operating, and desired maximum pressure drop from the cannula inlet to the pump inlets at the desired maximum system flow rate. The cannula 700 of the present example is substantially similar to the cannula 272 described above such that description of like features will not be repeated, and those features that are the same as above will be referenced with the same numbers used above, however features that are new or different will be assigned new reference numbers and described accordingly.

By way of example only, the expandable cannula 700 comprises an expandable body 702 having an expanded portion 703, a distal end 704, a proximal end 706, inner lumen 708, and one or more outlet port(s) 710 formed in the bottom portion of the body 702 (by way of example). The expandable body 702 comprises a thin-walled, self-expanding, re-collapsible tube made from flexible polymer and reinforcing frame, and includes an inner lumen 708 extending axially therethrough and a proximal taper 712 at the proximal end 706 of the expandable body 702. The inner lumen 708 is sized and configured to allow passage of a number of instrument and components therethrough, including but not limited to the obturator 218, guide wire 202, and the like, and is also configured to allow the flow of fluids therethrough.

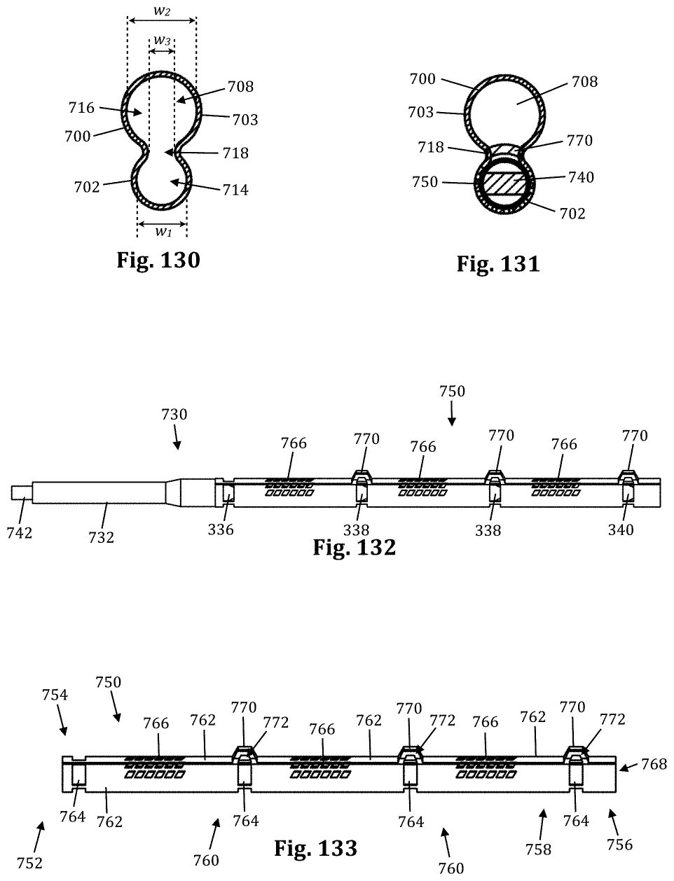

The inner lumen 708 is also configured to house the shroud 750, which itself contains one or more middle and/or distal pump assemblies 338, 340 that are inserted into the cannula 700 after expansion of the expandable body 702. An outlet port 710 is provided for each middle and/or distal pump assembly 338, 340 within the cannula 700 to allow the fluid to flow out of the cannula 700, bypassing the fluid inlet ports for the pump assemblies, which are inside the cannula 700. In some embodiments, a proximal outlet port 711 is provided in proximal pump housing 270, which is part of the catheter shaft 268 proximal of the proximal taper 712 to allow the fluid to egress from the proximal pump 336. In some embodiments, the inner lumen 708 may comprise a first portion 714 and a second portion 716 separated by a neck portion 718. When expanded, the first portion 714 may have a first width dimension w₁, the second portion 716 may have a second width dimension w₂ that is greater than the first width dimension to accommodate for bypass flow to proximal pumps (e.g. proximal pump 336 and/or middle pump(s) 338), and the neck portion 718 may have a width dimension w₃ that is less than the width dimension(s) of the first and second portions 714, 716, such that w₂>w₁>w₃. In some embodiments, the expanded cannula body 702 may have a “dumbbell” cross-sectional shape, as shown by way of example only in FIGS. 130-131. In some embodiments, the first portion 714 is configured to receive the shroud 750 therein. In some embodiments, the second portion 716 comprises the portion of the inner lumen 708 contained within the expanded portion 703 of the expandable body 702. In some embodiments, the decreased width of the neck portion 718 functions as an alignment feature that interacts with the expanded outlet partitions 770 of the shroud 750 (described below) to ensure that the outlet ports 764 of the shroud 750 and the pump assemblies 336, 338, 340 within the shroud 750 are properly aligned (e.g. axially and radially) with the outlet ports 710 upon insertion into the cannula 700.

In some embodiments, the cannula 700 may include one or more other pump alignment/location features previously described, such as a laterally-oriented pump stop and/or an anti-rotation feature.

By way of example only, FIGS. 132-136 illustrate an example of a pump assembly 730 according to some embodiments. By way of example, the pump assembly 730 may include a pump sheath 732, a pump drive assembly 740 (e.g., which includes a drive cable 742 and the impeller pumps 336, 338, 340), and a shroud 750. By way of example, the shroud 750 includes a proximal end 752 comprising a proximal shroud portion 754 and a distal end 756 comprising a distal shroud portion 758. In some embodiments, the shroud 750 further includes one or more middle shroud portions 760. In some embodiments, each of the shroud portions comprises a housing 762 configured to house the pump assemblies (e.g. pump bearings and impellers) therein. The housings 762 each include outlet ports 764 to enable blood to flow out of the pump assemblies. The outlet ports may be positioned radially about the housing in a 360° manner to maximize blood flow through the pumps. In some embodiments, the shroud 750 further includes several flexible, perforated segments 766 positioned between the housings 762 that function as inlet ports 766 to supply blood to the proximal and middle pumps 336, 338, and also to enable the shroud 750 to traverse vasculature as it is being inserted to a target site (e.g., a heart). The distal end 756 includes a distal inlet port 768 to supply blood flow to the distal pump 340.

By way of example, the middle shroud portions 760 and distal shroud portion 758 may each include an expandable outlet partition 770 covering (but not blocking) a portion of the outlet ports 764 to prevent outflow from the impellers from entering the second portion 716 of the inner lumen 708, thereby ensuring that the outflow from the impellers exits the cannula 700 through flow ports 710. Similar to the tubular pump guide 614 of cannula 600 above, the expandable outlet partitions 770 of the present example may be self-expanding to create a void 772 between the pump shroud 750 and the outlet partition 770 while fluidly separating the void 772 from the inner lumen 708. As previously described, the outlet partitions 770 are sized and configured to expand into the neck portion 718 of the cannula 700, to ensure proper alignment of the pump shroud 750 (and thus the pumps 336, 338, 340) within the cannula 770, for example such that the outlet ports 764 of the shroud 750 are aligned with flow ports 710, 711 of the cannula 700. Thus, the expandable outlet partitions 770 enable the use of a shroud 750 with a full 360° array of flow ports 764 because the void 772 enables fluid outflow through the flow ports 764 of the pump shroud 750 facing the partitions 770 and directs the outflow through the flow ports 710 of the cannula 700. Additionally, the flow ports 710 of the instant example may be formed not only on the “bottom” of the cannula 700 but also partially on the lateral sides. This example configuration is advantageous because it increases the flow rate of the middle and distal pumps 338, 340.

In some embodiments the shroud portions 754, 758, 760 are connected to enable simultaneous radial orientation with the cannula 770 to align the expandable outlet partitions 770 with the neck portion 718 of the cannula 770. By way of example, this connection may be accomplished by manufacturing the shroud 750 as a single unitary piece, or alternatively by manufacturing shroud segments individually and then connecting the segments together. In some embodiments, the pump sheath 732 and shroud 750 are connected to prevent rotation of the middle shroud portions 760 and/or distal shroud portions 758, ensuring consistent rotational torque allowing for motor pulsatility measurements indicating correct pump positioning in the ventricle (for example). In some embodiments, the distal pump bearings may be connected to the middle shroud portions 760 and/or distal shroud portions 758 so the torque of the middle and/or distal pumps 338, 340 does not cause middle and/or distal shroud portions 760, 758 to rotate.

In some embodiments, the cannula 700, location features, anti-rotation features, and/or outlet partitions 770 are elastically collapsible when pulled inside the delivery sheath and elastically expandable when pushed outside the delivery sheath. By of example only, the cannula body 700, location features, anti-rotation features, and/or outlet partitions 770 may be made of an elastic material (e.g., rubber, plastic, etc.) or a reinforced elastic material (e.g., nitinol, stainless steel, etc.). In some embodiments, the cannula 700, location features, anti-rotation features, and/or outlet partitions 770 may be made of dual-wall elastic material (e.g., rubber, plastic, etc.) having an internal space or void configured to receive pressurized fluid (e.g., saline, blood compatible gas, etc.) to facilitate expansion and/or contraction of the feature for delivery and/or removal into or out of the patient through the delivery sheath.

By way of example, the proximal taper 712 funnels the fluid (e.g., blood) flow within the inner lumen 708 to the inlet of the proximal pump 336 and facilitates collapsing of the cannula 700 for removal from the body, as described above with respect to the proximal tapers of previously described cannula embodiments.

In some embodiments, the cannula 700 of the present example may be configured for use with a proximal pump housing or shroud 754 including a flow port 711 that is part of, or connected to, the catheter shaft 268, for example like the proximal pump assembly 336 described above and shown in FIG. 126. In some embodiments, the shroud 750 may be positioned relative to the catheter shaft 268 such that the proximal-most outlet port 764 is aligned with the flow port 711 of the cannula 700. In some embodiments, the cannula 700 of the present example may be configured for use with a proximal pump housing that is part of the pump assembly (for example like the proximal pump assembly 418 described above). In some embodiments, the cannula 700 and proximal guide shaft may be formed as one piece back to the hemostasis valve, or alternatively may be formed of two or more pieces.

Any of the features or attributes of the above the above-described embodiments and variations can be used in combination with any of the other features and attributes of the above-described embodiments and variations as desired.

From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by any and all claims deriving from this disclosure when interpreted in accordance with the benefit to which they are fairly, legally, and equitably entitled.

What is claimed is:

1. A fluid pump system, comprising:
a first pump assembly having a fluid inlet port, a fluid outlet port, and an impeller;
a second pump assembly having a fluid inlet port, a fluid outlet port, and an impeller;
a catheter configured to deliver the first and second pump assemblies to a target site, the catheter having a distal end comprising a cannula, the cannula having a single lumen extending proximally from an inlet port of the cannula and configured to supply fluid to the inlet ports of each of the first and second pump assemblies, the cannula further having a pump guide configured to receive the second pump assembly therein;
wherein the first pump assembly and second pump assembly are arranged physically in series in a collinear manner along a longitudinal axis with the first pump assembly located proximally relative to said second pump assembly;
wherein the first pump assembly and second pump assembly operate simultaneously and flow functionally in parallel such that fluid exiting the outlet port of the first pump assembly bypasses the inlet port of the second pump assembly.

2. The fluid pump system of claim 1, wherein the cannula has an expandable body and an internal space, and the cannula is configured to be expandable from a first, collapsed configuration in which the expandable body occupies the internal space to a second, expanded position wherein the expandable body increases the volume of the internal space.

3. The fluid pump system of claim 2, wherein the second pump assembly is positioned within the cannula in the volume of internal space previously occupied by the collapsed cannula body.

4. The fluid pump system of claim 2, wherein the cannula is configured to be positioned within the target site first, and the first and second pump assemblies are configured to be introduced to the target site through the catheter thereafter.

5. The fluid pump system of claim 1, wherein the pump guide is tubular.

6. The fluid pump system of claim 1, wherein the pump guide is form-fitting such that the second pump assembly fits snugly within the pump guide.

7. The fluid pump system of claim 6, wherein the second pump assembly has a plurality of fluid outlet ports distributed about a circumference of the fluid pump assembly, the cannula has a fluid outlet port in alignment with at least one of the fluid outlet ports of the second pump assembly, and the form-fitting pump guide prevents fluid flow from at least one of the plurality of fluid outlet ports of the second pump assembly into the lumen of the cannula.

8. The fluid pump system of claim 1, wherein the pump guide includes a self-expanding internal partition that expands to create a void between the pump shroud and internal partition while fluidly separating the void from the lumen of the cannula.

9. The fluid pump system of claim 8, wherein the second pump assembly has a plurality of fluid outlet ports distributed about a circumference of the fluid pump assembly, the cannula has at least one fluid outlet port in alignment with the fluid outlet ports of the second pump assembly, and the self-expanding internal partition directs fluid flow from the second pump assembly through the void to the at least one fluid outlet port of the cannula.

10. The fluid pump system of claim 1, further comprising a third pump assembly arranged collinearly with the first and second pump assemblies along the longitudinal axis, the third pump assembly having a fluid inlet port, a fluid outlet port, and an impeller, wherein fluid exiting the outlet port of the first pump assembly bypasses the inlet ports of the second and third pump assemblies, and fluid exiting the outlet port of the second pump assembly bypasses the inlet port of the third pump assembly.

11. The fluid pump system of claim 10, wherein the cannula further includes a second pump guide configured to receive the third pump assembly therein.

12. The fluid pump system of claim 11, wherein the second pump guide is tubular.

13. The fluid pump system of claim 11, wherein the second pump guide is form-fitting such that the third pump assembly fits snugly within the second pump guide.

14. The fluid pump system of claim 13, wherein the third pump assembly has a plurality of fluid outlet ports distributed about a circumference thereof, the cannula has a second fluid outlet port in alignment with at least one of the fluid outlet ports of the third pump assembly, and the second form-fitting pump guide prevents fluid flow from at least one of the plurality of fluid outlet ports of the third pump assembly into the lumen of the cannula.

15. The fluid pump system of claim 11, wherein the second pump guide includes a self-expanding internal partition that expands to create a void between the pump shroud and internal partition while fluidly separating the void from the lumen of the cannula.

16. The fluid pump system of claim 15, wherein the third pump assembly has a plurality of fluid outlet ports distributed about a circumference of the fluid pump assembly, the cannula has at least one fluid outlet port in alignment with at least one of the fluid outlet ports of the third pump assembly, and the self-expanding internal partition directs fluid flow from the third pump assembly through the void to the at least one fluid outlet port of the cannula.

17. The fluid pump system of claim 1, further comprising a motor assembly and a drive cable, wherein the drive cable is rotatably connected to the motor assembly, the impeller of the first pump assembly, and the impeller of the second pump assembly, and wherein activating the motor assembly causes the first and second pump assemblies to operate simultaneously.

18. The fluid pump system of claim 1, wherein the cannula further includes an anti-rotation feature configured to prevent the second pump assembly from rotating relative to the cannula when the second pump assembly is positioned within the cannula.

19. The fluid pump system of claim 18, wherein the anti-rotation feature is an axially aligned elongated rail.

20. The fluid pump system of claim 19, wherein the second pump assembly includes an axially aligned groove configured to mate with the axially aligned elongated rail.
